(19) United States
(12) Patent Application Publication
(10) Pub. No.: US 2024/0266945 A1
(43) Pub. Date: Aug. 8, 2024

(54) APPARATUS FOR SUPPLYING POWER AND MEDICAL DEVICE

(71) Applicant: SHANGHAI UNITED IMAGING HEALTHCARE CO., LTD., Shanghai (CN)

(72) Inventors: Yansi YANG, Shanghai (CN); Haike WANG, Shanghai (CN); Hongtao WANG, Shanghai (CN); Xiaohua QIN, Shanghai (CN); Fengjie SUN, Shanghai (CN); Ruijian WEN, Shanghai (CN)

(73) Assignee: SHANGHAI UNITED IMAGING HEALTHCARE CO., LTD., Shanghai (CN)

(21) Appl. No.: 18/616,220

(22) Filed: Mar. 26, 2024

Related U.S. Application Data
(63) Continuation of application No. PCT/CN2022/125870, filed on Oct. 18, 2022.

(30) Foreign Application Priority Data
Oct. 19, 2021 (CN) 202111213870.3
Nov. 12, 2021 (CN) 202111339821.4

(51) Int. Cl. H02M 1/12; A61B 8/00; H02M 1/14; H02M 3/158

(52) U.S. Cl. CPC H02M 1/126; A61B 8/56; H02M 1/143; H02M 3/158

(57) ABSTRACT

An apparatus for supplying power is disclosed. The apparatus may comprise a power supply circuit and a processing unit, wherein the power supply circuit may be connected with an electric energy input end of the processing unit, and the power supply circuit may provide a first power supply for the processing unit; and a forward voltage output end of the processing unit may provide a forward power supply, and a backward voltage output end of the processing end may provide a negative power supply, wherein the processing unit may include a resonant circuit, a first switching frequency of the processing unit may include a resonant frequency of the resonant circuit, and the resonant frequency may be outside an effective frequency range of a load of the apparatus for supplying power.

### CROSS-REFERENCE TO RELATED APPLICATIONS

This application is a Continuation of International Application No. PCT/CN2022/125870, filed on Oct. 18, 2022, which claims priority to Chinese Patent Application No. 202111339821.4, filed on Nov. 12, 2021, and Chinese Patent Application No. 202111213870.3, filed on Oct. 19, 2021, the contents of each of which are incorporated herein by reference in their entirety.

### TECHNICAL FIELD

The present disclosure relates to the field of power supply, in particular, to an apparatus for supplying power and a medical device.

### BACKGROUND

In medical field, the operation of some medical devices requires a high-voltage power supply provided by an apparatus for supplying power. For example, an ultrasound device usually requires a positive and negative high-voltage power supply to generate pulses for subsequent detection. As another example, an energy spectrum detector requires a high-voltage field strength provided by the high-voltage power supply to accelerate electrons to be collected for the subsequent energy spectrum imaging.

However, in the current technique, the output electrical energy of the power supply apparatus is usually unstable due to various disturbances, which affects the operating quality of the load. For example, a signal in the power supply apparatus may cause electromagnetic interference (EMI) to a detector easily, which affects the accuracy of the detection. As another example, the high-voltage power supply needs to turn off and on a switching element repeatedly, which is easy to generate an alternating component, resulting in a high ripple in the output electrical energy and an unstable load operation. As a further example, with the increase of the load current, the losses of transformers, capacitors, and other components in the high-voltage power supply may also increase, which causes a voltage drop of a high-voltage direct current (DC) signal easily, and affects the operation of the load.

Therefore, it is desirable to provide an apparatus for supplying stable high-voltage power.

### SUMMARY

One aspect of the present disclosure may provide an apparatus for supplying power, comprising a power supply circuit and a processing unit, wherein the power supply circuit may be connected with an electric energy input end of the processing unit, and the power supply circuit may provide a first power supply for the processing unit; and a forward voltage output end of the processing unit may provide a forward power supply, and a backward voltage output end of the processing end may provide a negative power supply, wherein the processing unit may include a resonant circuit, a first switching frequency of the processing unit may include a resonant frequency of the resonant circuit, and the resonant frequency may be outside an effective frequency range of a load of the apparatus for supplying power.

Another aspect of the present disclosure may provide a high-voltage power supply, comprising: a booster circuit and a compensation circuit, wherein an input end of the booster circuit may be connected with an input power supply, and an output end of the booster circuit may be configured to provide output electrical energy; an input end of the compensation circuit may be at least connected with the input end of the booster circuit; an output end of the compensation circuit may be at least connected with a reference end of the booster circuit, and the compensation circuit is configured to adjust the output electrical power of the booster circuit according to a voltage of the input power supply.

The processing unit provided by the embodiments of the present disclosure may convert the first power supply into the forward power supply and the negative power supply, so that the forward power supply and the negative power supply that satisfy certain conditions may be provided simultaneously. Further, by setting the resonant frequency of the resonant circuit in the processing unit outside an effective frequency range, the signal and the output electrical energy processed by the power supply apparatus during operation may avoid affecting the operation of the load within the effective frequency range, which can reduce electromagnetic interference (EMI) of the power supply to the load and improve the stability and accuracy of the load operation.

Further, the embodiments of the present disclosure adjust the output electric energy of the booster circuit by the compensation circuit to avoid a voltage drop of the electrical energy. Ripple can be filtered by using the filtered circuit to provide a low-ripple and stable high-voltage output electrical energy.

### BRIEF DESCRIPTION OF THE DRAWINGS

The present disclosure is further illustrated in terms of exemplary embodiments, and these exemplary embodiments are described in detail with reference to the drawings. These embodiments are not restrictive. In these embodiments, the same number indicates the same structure, wherein:

FIG. 1 is a schematic diagram illustrating a structure of an apparatus for power supply according to some embodiments of the present disclosure;

FIG. 2 is a schematic diagram illustrating a topography of a supply power according to some embodiments of the present disclosure;

FIGS. 3A and 3B are schematic diagrams illustrating a circuit structure of a supply power according to some embodiments of the present disclosure;

FIG. 4 is a schematic diagram illustrating a circuit structure of a processing unit according to some embodiments of the present disclosure;

FIG. 5 is a schematic diagram illustrating a relationship between frequency and current according to some embodiments of the present disclosure;

FIG. 6 is a schematic diagram illustrating a simplified structure of a resonant circuit according to some embodiments of the present disclosure;

FIG. 7 is a schematic diagram illustrating a structure of a processing unit according to some embodiments of the present disclosure;

FIG. 8 is a schematic diagram illustrating a structure of a driving circuit according to some embodiments of the present disclosure;

FIG. 9 is a schematic diagram illustrating a circuit structure of a driving circuit according to some embodiments of the present disclosure;

FIG. 10 is a schematic diagram illustrating a structure of a switching circuit according to some embodiments of the present disclosure;

FIG. 11 is a schematic diagram illustrating a structure of a feedback circuit according to some embodiments of the present disclosure;

FIGS. 12A and 12B are schematic diagrams illustrating a structure of a voltage sampling circuit according to some embodiments of the present disclosure;

FIG. 13 is a schematic diagram illustrating a structure of a power supply monitoring module according to some embodiments of the present disclosure;

FIGS. 14A and 14B are schematic diagrams illustrating a circuit structure of a comparison circuit according to some embodiments of the present disclosure;

FIG. 15 is a schematic diagram illustrating a generation manner of a reference voltage according to some embodiments of the present disclosure;

FIG. 16 is a schematic diagram illustrating a circuit structure of a reference voltage circuit according to some embodiments of the present disclosure;

FIG. 17 is a schematic diagram illustrating a structure of a medical apparatus according to some embodiments of the present disclosure;

FIG. 18 is a schematic diagram illustrating a structure of a medical imaging apparatus according to some embodiments of the present disclosure;

FIG. 19 is a schematic diagram illustrating a circuit structure of a high-voltage power supply according to some embodiments of the present disclosure;

FIG. 20 is a schematic diagram illustrating a structure of a high-voltage power supply according to some embodiments of the present disclosure; and

FIG. 21 is a schematic diagram illustrating a circuit structure of a high-voltage power supply according to some embodiments of the present disclosure.

### DETAILED DESCRIPTION

In order to illustrate the technical solutions related to the embodiments of the present disclosure, brief introduction of the drawings referred to in the description of the embodiments is provided below. Obviously, drawings described below are only some examples or embodiments of the present disclosure. Those having ordinary skills in the art, without further creative efforts, may apply the present disclosure to other similar scenarios according to these drawings. Unless stated otherwise or obvious from the context, the same reference numeral in the drawings refers to the same structure and operation.

It will be understood that the terms “system,” “device,” “unit,” and/or “module” used herein are one method to distinguish different components, elements, parts, sections, or assemblies of different levels in ascending order. However, the terms may be displaced by other expressions if they may achieve the same purpose.

As shown in the present disclosure and claims, unless the context clearly indicates exceptions, the words “a,” “an,” “one,” and/or “the” do not specifically refer to the singular, but may also include the plural. The terms “including” and “comprising” only suggest that the steps and elements that have been clearly identified are included, and these steps and elements do not constitute an exclusive list, and the method or device may also include other steps or elements.

A flowchart is used in the present disclosure to illustrate the operation performed by the system according to the embodiment of the present disclosure. It should be understood that the preceding or subsequent operations are not necessarily performed accurately in sequence. Instead, the steps may be processed in reverse order or simultaneously. At the same time, other operations may add to these procedures, or remove one or more operations from these procedures.

An apparatus for supplying power provided by one or more embodiments of the present disclosure may provide a relatively high electrical energy, which may be convenient for applying to various business scenarios that require high-voltage electrical energy, such as a generation scenario that uses a forward high-voltage electrical energy and a backward high-voltage electrical energy to generate signals such as pulses or lasers, a detection scenario that uses a high-voltage field strength provided by the high-voltage power supply to collect signals or particles, or a classification scenario that uses the high-voltage field strength to perform gas purification, liquid recovery, impurity removal, etc. In some alternative embodiments, the power supply apparatus may provide a forward high-voltage electrical energy or a backward high-voltage electrical energy for the ultrasonic device to generate the pulses for subsequent detection. In some alternative embodiments, the power supply apparatus may provide a high-voltage field strength provided by an energy spectrum detector of a medical imaging device to collect required electrons for the energy spectrum detector. In some embodiments, an input electrical energy of the power supply apparatus may be a direct current (DC) signal or an alternating current (AC) signal, and the input electrical energy of the apparatus may be selected according to the types of high-voltage power supply.

In some embodiments, the power supply apparatus may include a processing unit, the processing unit may boost the electrical energy input from external source to provide high-voltage electrical energy for the load. However, the signal and the output electrical energy processed by the power supply apparatus during operation may be prone to electromagnetic interference (EMI), which may affect the operating of the load and reduce the operating quality of the load. For example, harmonics in the signal of the power supply apparatus and the power supply provided by the power supply apparatus may cause the EMI to the ultrasound device through the power supply and affect the quality of the signal generated by the ultrasound device when supplying the power.

Further, with the increase of the load current, the losses of transformers, capacitors, and other components in the high-voltage power supply may also increase, which causes a voltage drop of a high-voltage direct current (DC) signal easily, and affects the operation of the load. When the output of the high-voltage power supply is unstable, the energy spectrum detector may not stably collect the required electrons, which may also affect the imaging quality.

The power supply apparatus provided by the embodiments of the present disclosure, by controlling the resonant frequency of the resonant circuit, the signal processed by the power supply apparatus, and the forward power supply and the negative power supply supplies provided by the power supply apparatus during the operation may avoid affecting the operation of the load within the effective frequency range, which may reduce the EMI of the power supply to the load and improve the operating quality of the load. At the same time, the power supply apparatus provided by the embodiments of the present disclosure may adjust the output of the booster circuit by the compensation circuit to avoid the voltage drop, and ripple may be filtered by using the filtered circuit to provide a low-ripple and stable high-voltage output electrical energy.

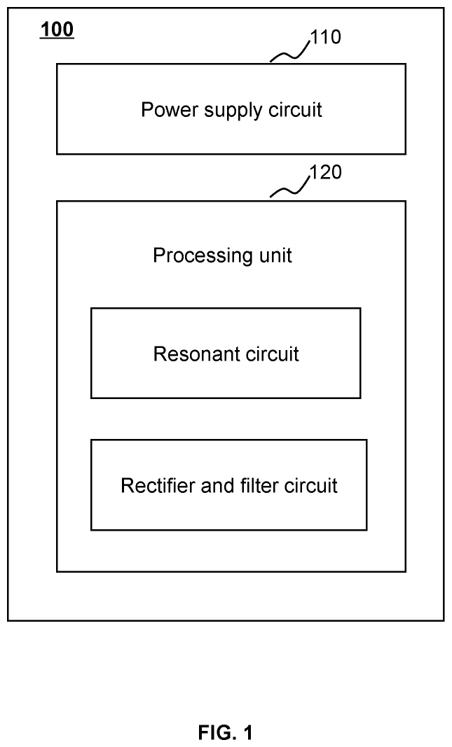

FIG. 1 is a schematic diagram illustrating a structure of an apparatus for power supply according to some embodiments of the present disclosure. As shown in FIG. 1, an apparatus 100 for power supply may include a power supply circuit 110 and a processing unit 120.

The power supply circuit 110 may be connected with an electrical input end of the processing unit 120 and configured to provide a first power supply for the processing unit 120. In some embodiments, the first power supply may be used to provide the power for the processing unit 120, and the processing unit 120 may convert the power supply to supply the power for the load. In some embodiments, the first power supply may be the direct current (DC) power supply or the alternating current (AC) power supply. In some embodiments, the power supply circuit 110 may further a filter circuit configured to filter out interference on a power supply bus.

In some embodiments, the power supply circuit 110 may supply the power for active electronics in the processing unit 120, since these active electronics have the highest operating efficiency under a specific voltage, the power supply circuit 110 may further include a voltage converter configured to convert a power supply voltage provided for the active electronics into the required voltage, to improve the operating efficiency.

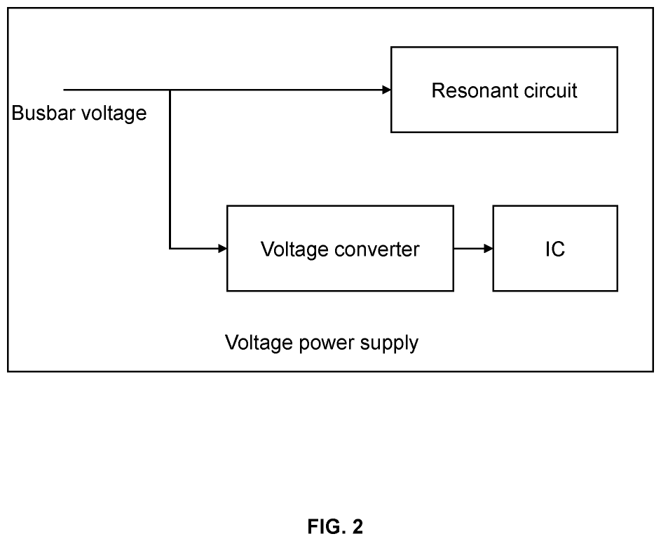

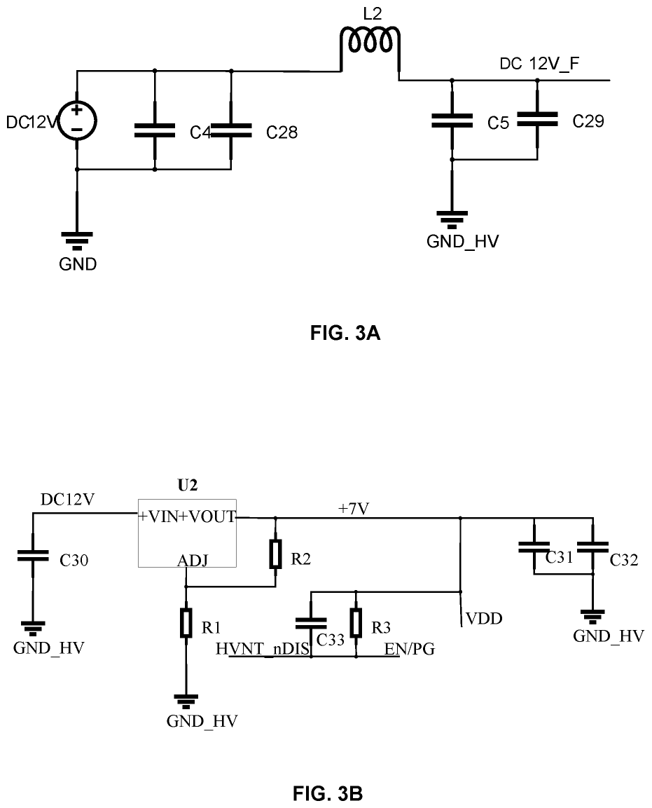

FIG. 2 is a schematic diagram illustrating a topography of a supply power according to some embodiments of the present disclosure. As shown in the power supply topography of FIG. 2, on the one hand, a busbar voltage of the power supply circuit 110 may provide the resonant circuit of the processing unit 120 by the filter circuit. On the other hand, the busbar voltage of the power supply circuit may provide an integrated circuit (IC) device by the voltage converter. More descriptions of the specific implementation manner of the power supply circuit 110 may be found in FIG. 3A, FIG. 3B, and the related descriptions, which may not be repeated here.

The processing unit 120 may be used to convert the first power supply provided by the power supply circuit 110 into the forward power supply and the negative power supply. In some embodiments, the processing unit 120 may include a forward output end and a backward output end, wherein the forward output end may be used to provide the forward power supply for the load, and the backward output end may be used to provide the negative power supply for the load.

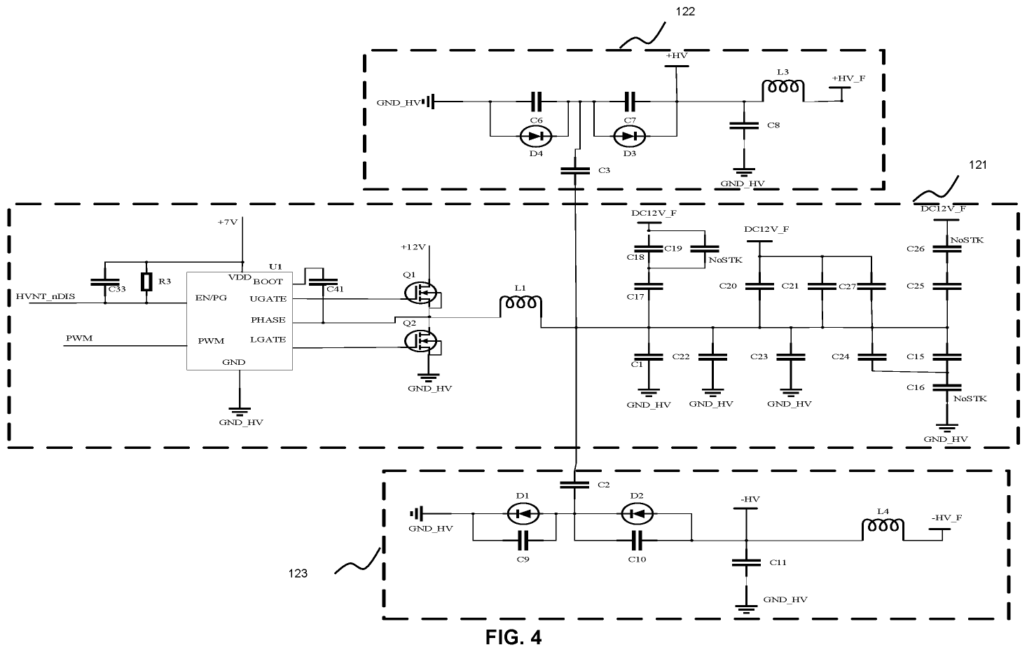

In some embodiments, the processing unit 120 may include a resonant circuit and a rectifier and filter circuit, and a first switching frequency of the processing unit 120 may include a resonant frequency f of the resonant circuit. The resonant circuit may be used to adjust the resonant frequency f and output a resonant signal, and the rectifier and filter circuit may be used to rectify and filter the resonance signal to output the forward power supply and the negative power supply. In some embodiments, an input end of the resonant circuit may be connected with the power supply circuit 110 to receive the first power supply, and an output end of the resonant circuit may be connected with an input end of the rectifier and filter circuit to output the resonant signal. In some embodiments, the rectifier and filter circuit may be symmetrically arranged with the resonant circuit as a center, to make a symmetry degree of the forward power supply and the negative power supply output by the rectifier and filter circuit within a preset symmetry range. More descriptions of the specific implementation manner of the resonant circuit and the rectifier and filter circuit may be found in FIGS. 4-6 and the related descriptions, which may not be repeated here.

In some embodiments, the processing unit 120 may further include a booster circuit, and an input end of the booster circuit may be connected with an output end of the power supply circuit 110. A voltage output by the booster circuit may be higher than a voltage of the first power supply.

The booster circuit may be a circuit used for boosting the voltage. In some embodiments, the booster circuit may boost the voltage of the first power supply to output a high-voltage electrical energy. In some embodiments, the booster circuit may be a circuit such as a single ended primary inductor converter (Sepic), a LC resonant circuit, a Flyback circuit, etc., which may not be repeated here.

In some embodiments, the first switching frequency of the processing unit 120 may further include a resonant frequency f of the booster circuit, the resonant frequency f of the booster circuit may be outside an effective frequency range of the load, and the resonant frequency f may be outside an nth harmonic frequency range of the effective frequency range of the load. Further, in some embodiments, the nth harmonic range of the resonant frequency f may be outside an effective frequency range of the load, and the nth harmonic range of the resonant frequency f may be outside the nth harmonic frequency range of the effective frequency range of the load.

In some embodiments, the first switching frequency may include turn-on and turn-off frequencies of the components in the processing unit and a frequency at which resonance occurs. In some embodiments, the resonant frequency may be a frequency at which resonance of the booster circuit occurs, which may be an inherent property of the circuit. When the resonant frequency f of the booster circuit is outside an effective frequency range [a, b] of the load, the power supply apparatus 100 may avoid affecting the operation of the load within the effective frequency range, which may reduce the EMI of the power supply to the load and improve the operating quality of the load. More descriptions of the specific implementation manner of the resonant frequency and the effective frequency range may be found in the following descriptions of the resonant circuit, which may not be repeated here.

In some embodiments, the power supply apparatus 100 may further include a drive circuit. The drive circuit may be connected with the processor and the processing unit 120 and configured to output a drive signal to the processing unit 120 according to instructions transmitted from the processor, so as to control the output of the processing unit 120. In some embodiments, the drive circuit may be a pulse width modulation (PWM) drive circuit and may be used to output a PWM signal.

In some embodiments, the power supply apparatus 100 may further include a processor. The processor may be an integrated circuit that performs a processing business. For example, the processor may be a single chip microcomputer (MCU), a Complex Programmable logic device (CPLD), a Field Programmable Gate Array (FPGA), or one or more combinations thereof. In some embodiments, the processor may be an internal processor of the power supply apparatus 100 or an external processor of the power supply apparatus 100, and may be used to remote control the power supply apparatus 100. It should be noted that the processor and the processing unit 120 may be different circuit devices, the processing unit 120 may be used to convert the first power supply into the forward power supply and the negative power supply, and the processor may be used to issue instructions to the processing unit 120 to control the output of the processing unit 120.

The load may be an electrical device that consumes electrical energy. Further, in some embodiments, the load may be a device that requires stable forward and backward high-voltage electrical energy. In some embodiments, the load may be an output device that uses the forward and backward high-voltage electrical energy to generate signals such as pulses, lasers, etc. For example, the load may be an ultrasound system component, such as an ultrasound probe, an ultrasound diagnostic, etc., since an ultrasound signal is relatively small and easy to be interfered by other signals, the quality of high-voltage power supply may be required to be high and a good EMI environment may be required. In some embodiments, the load may be a detection device that uses the forward and backward high-voltage electrical energy to collect signals or particles, such as an energy spectrum detector, a ray detector, a sound acquisition system, etc.

FIGS. 3A and 3B are schematic diagrams illustrating a circuit structure of a supply power according to some embodiments of the present disclosure.

In some embodiments, the power supply circuit may include a first power supply circuit and a second power supply circuit. The first power supply circuit may be used to provide a first power supply for the processing unit 120, and the processing unit 120 may convert the first power supply into the forward power supply and the negative power supply to supply the power for the load. The second power supply circuit may be used to provide a power supply voltage for the active electronic devices in the processing unit 120. In some embodiments, the second power supply circuit may be used to provide the power supply voltage for a driver U1 of the processing unit 120.

As shown in FIG. 3A, the first power supply circuit may be used to provide a first power supply for the processing unit 120. In some embodiments, the first power supply circuit may include a first energy source configured to provide the first power supply, one end of the first energy source may be connected in series with a first end of an inductor L2, and another end of the first energy source may be grounded. A second end of the inductor L2 may be an output end of the first power supply circuit and configured to output the first power supply to the processing unit 120, such as a switch of the processing unit 120. In some embodiments, the first energy source may be a DC power supply, such as a 12V DC power supply. In some embodiments, the inductor L2 may be replaced by a noise suppression component such as a magnetic bead.

In some embodiments, as shown in FIG. 3A, filter capacitors C4 and C28 may be connected in parallel with both ends of a first DC source, and one end of filter capacitors C5 and C29 may be connected in parallel with a second end of the inductor L2, and another end of the filter capacitors C5 and C29 may be grounded in parallel. The above inductor L2, filter capacitors C4, C5, C28, and C29 may form a TT-type filter circuit and may be configured to filter interference of a DC bus. In some embodiments, capacitances of the filter capacitors C4 and C28 may be different and capacitances of filter capacitors C5 and C29 may be the same or different, which may filter out the noise interference in different frequency bands. In some embodiments, the filter capacitors C5 and C29 may be implemented by two or more filter capacitors.

As shown in FIG. 3B, the second power supply circuit may be used to provide a second power supply for a driver U1 of the processing unit 120. In some embodiments, the second power supply may include a second energy source configured to provide a second power supply, one end of the second energy source may be grounded by a capacitor C30, and another end of the second energy source may be connected with an input end of a voltage converter U2, an output end of the voltage converter U2 may be connected with an electrical energy input end of the driver U1 to provide a rated operating voltage for the driver U1. In some embodiments, the second energy source may be a DC power supply, such as a 12V DC power supply. If the driver U1 has the highest efficiency when the power supply voltage is 7˜8V, the voltage converter U2 may convert the 12V DC power supply into a DC power supply within 7V-8V to supply the power for the driver U1. In some embodiments, the output voltage of the voltage converter U2 may be adjusted according to the power supply voltage required by different models of the driver U1.

In some embodiments, the output end of the voltage converter U2 may be connected with an adjustment end of the voltage converter U2 by a resistor R2, an adjustment end of the voltage converter U2 may be grounded by a resistor R1, the resistor R1 and the resistor R2 may be used to adjust the output voltage of the voltage converter U2. In some embodiments, the voltage converter U2 may be a module with voltage conversion, such as a low dropout regulator (LDO), a DCDC, or other power supply modules.

In some embodiments, the power supply circuit 110 may further include a third power supply circuit, a fourth power supply circuit, a fifth power supply circuit, . . . , and circuits similar in structure to the second power supply circuit, which may be used to provide the power supply voltage (e.g., a biasing voltage, a third power supply, a fourth power supply) of other active electronic devices, such as an operational amplifier in a voltage sampling circuit, a comparator in a comparison circuit, a voltage buffer U3 in the drive circuit, etc.

Taking the booster circuit as the resonant circuit as an example, the specific implementation manner of the boosting process and controlling the resonant frequency may be described.

FIG. 4 is a schematic diagram illustrating a circuit structure of a processing unit according to some embodiments of the present disclosure.

In some embodiments, the above processing unit 120 may include a resonant circuit 121, the resonant circuit 121 may include a resonant capacitor bank and an inductor, the resonant capacitor bank may include one or more resonant capacitors, the resonant capacitor bank may be connected in series with the inductor, the resonant capacitor bank, and the inductor may be used to adjust the resonant frequency f.

The resonant circuit 121 may be a port network exhibiting capacitive, inductive, and resistive properties, which may generate alternating electrical energy. In some embodiments, when a frequency of the input drive signal (i.e., the control signal) is equal to the resonant frequency of the resonant circuit 121, the resonant circuit 121 may be pure resistance; when the frequency of the input drive signal is less than the resonant frequency of the resonant circuit 121, the resonant circuit 121 may be capacitive; when the frequency of the input drive signal is greater than the resonant frequency of the resonant circuit 121, the resonant circuit 121 may be inductive. In some embodiments, the resonant circuit 121 may convert the DC power supply into the AC power supply by the functions of the energy storage and the conversion of the resonant capacitor and inductor, so as to improve the booster and change a current direction of the resonant circuit 121. As shown in FIG. 4, the resonant circuit 121 may include the resonant capacitor bank (e.g., capacitors C1 and C15-C27 as shown in FIG. 4) and the inductor (e.g., an inductor L1 as shown in FIG. 4), when the resonant capacitor bank charges, the current may flow from the inductor to the resonant capacitor; when the resonant capacitor bank discharges, the current may flow from the resonant capacitor bank to the inductor.

The resonant frequency may be a frequency at which the resonant circuit 121 resonates, which may be an inherent property of the resonant circuit 121. When a signal frequency (i.e., a frequency of current commutation) of the resonant circuit 121 is the resonant frequency, the resonant frequency may resonate and current of the resonant circuit 121 may be close to a peak value. In some embodiments, the first switching frequency of the processing unit 120 may include the resonant frequency f of the resonant circuit 121.

In some embodiments, the resonant frequency f may be controlled by the resonant capacitor and inductor of the resonant circuit 121. In some embodiments, based on a resonant frequency equation of the resonant circuit 121, by adjusting the parameters of the resonant capacitor C and the inductor L, the resonant frequency f of the resonant circuit 121 may be adjusted. In some embodiments, the resonant frequency equation may be the following Equation (1):

$$f = \frac{1}{2 π \sqrt{LC}} \qquad ( 1 )$$

wherein f denotes the resonant frequency of the resonant circuit, L denotes an inductance value of the inductor, and C denotes a capacitance value of the resonant capacitor bank.

In some embodiments, the resonant capacitor bank may include one or more resonant capacitors and one or more inductors. In some embodiments, the inductance value L of the inductor may be adjusted by adjusting the parameters of the inductor L1. In some embodiments, by adjusting the count, one or more of the parameters, and the connection manners of capacitors C1 and C15-C27, the capacitance value C of the resonant capacitor bank may be adjusted. A combination manner of the inductor L1 and the resonant capacitor bank C may be described by taking FIG. 4 as an example.

As shown in FIG. 4, the inductor may include an inductor L1, the resonant capacitor bank may include capacitors C1 and C15-C27. The capacitor C1 may be connected in parallel with the capacitors C15-C27, one end of the capacitor C1 may be connected with the inductor L1, and another end of the capacitor C1 may be grounded. The capacitor C15 may be connected in series with the capacitor C16, the capacitor C15 may be connected in parallel with the capacitor C24, and one end of the capacitor C16 may be grounded. One end of the capacitor C16 away from the capacitor C15 may be connected with the inductor L1. The capacitor C17 may be connected in series with the capacitor C18, the capacitor C19 may be connected in parallel with the capacitor C18, one end of the capacitor C18 may be connected with the biasing voltage, and one end of the capacitor C18 away from the capacitor C17 may be connected with the inductor L1. The capacitor C20 and the capacitor C21 may be connected in parallel with the capacitor C27, one end of the capacitor C20 may be connected with the inductor L1, and another end of the capacitor C20 may be connected with the biasing voltage. The capacitor C25 may be connected in series with the capacitor C26, one end of the capacitor C25 may be connected with the inductor L1, and one end of the capacitor C26 may be connected with the biasing voltage.

It should be noted that, since volume of the inductor is generally larger than volume of the capacitor, in some embodiments, a relatively small inductor may be selected to keep the volume of the inductor. In some embodiments, in order to ensure that ripple of the power supply device is relatively small, the inductance value of the inductor may be selected according to the change rate of the current and the energy storage of the inductor. In some embodiments, an inductance value range of the inductor may be 10 nH˜10 uH.

In some embodiments, the output end of the resonant circuit 121 may include a connection point of the resonant capacitor bank and the inductor. The connection point is configured to output the resonant signal, and the voltage of the resonant signal may be greater than the voltage of the first power supply. In order to provide the forward and negative power supplies, in some embodiments, the resonant signal may be an AC signal, so that the rectifier and filter circuit may be used for processing to output the forward and negative power supplies. In some embodiments, the resonant signal may be a single frequency sine signal.

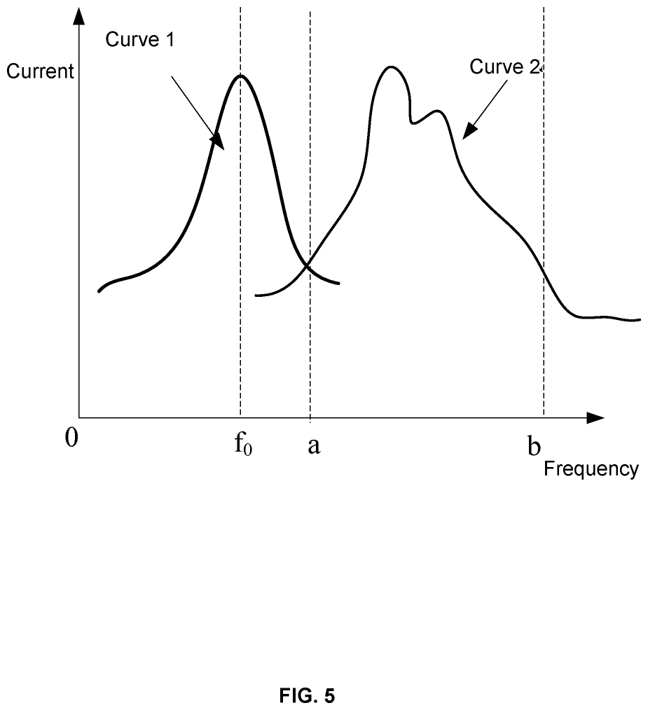

FIG. 5 is a schematic diagram illustrating a relationship between frequency and current according to some embodiments of the present disclosure.

In some embodiments, a relationship curve of a signal frequency of an input drive signal of the resonant circuit 121 and current of the resonant circuit 121 may be a curve 1 in FIG. 5. Since the input drive signal is equal to the resonant frequency, the resonant circuit 121 may be equivalent to a pure resistive port circuit, the current of the resonant circuit 121 has a maximum current, and the output power may be maximum. As shown in the curve 1, when the input drive signal of the resonant circuit 121 is at the resonant frequency f₀, the current of the resonant circuit 121 is close to the peak value, which may make the signal strength relatively large and reduce the interference of other signals. In some embodiments, a relationship curve of a frequency of a load signal and current of the load may be a curve 2 in FIG. 5. In some embodiments, an effective frequency of the load may be [a, b], wherein a denotes a first effective frequency, b denotes a second effective frequency. a may be less than or equal to b, to make [a, b] forms a range or specific v2losure. When the frequency of the load signal is within [a, b], the load current may be relatively large to ensure that signal-to-noise ratio of a signal acquisition system where the load current is located is maximum.

The load signal may be a signal involved in the operating of the load, which may include the signal and the processing signal obtained by the load during the operation, and the effective frequency range of the load may be a frequency range of most signals involved in the load during the ope2 Hz-20 KHz; when the load is an ultrasonic diagnostic apparatus, a frequency range of the ultrasonic diagnostic apparatus may be 2 MHz-20 MHz; when the load is an ultrasonic probe, an effective frequency range that the ultrasound probe can receive may be 2 MHz-6 MHz. The effective frequency range of the load in the present disclosure may not be limited here.

In some embodiments, the resonant frequency f of the resonant circuit 121 may be outside the effective frequency range [a, b]. As shown in FIG. 5, when the resonant frequency f₀ of the resonant circuit 121 is located on the left side of the effective frequency range [a, b], in the frequency range [a, b], the load current corresponding to the curve 2 may be greater than the current of the resonant circuit 121 corresponding to the curve 1. It should be noted that, in the frequency range [a, b], compared with the load current, the current of the resonant circuit 121 may be relatively small, and the signal strength of the resonant circuit 121 may be relatively small.

That is to say, by adjusting the resonant frequency f of the resonant circuit 121 to be outside the effective frequency range of the load, in the frequency range [a, b], for the signal of the load, the relatively small signal strength of the resonant circuit 121 may avoid affecting the operation of the load, so as to reduce the EMI of the power supply apparatus 100 to the load and improve the operating quality of the load.

In some embodiments, the nth harmonic of the resonant frequency f may be outside the effective frequency range [a, b] of the load. In some embodiments, the resonant frequency f may be outside an nth harmonic frequency range of the effective frequency range

$$[ \frac{( n + 1 ) a + ( n - 1 ) b}{2} , \frac{( n - 1 ) a + ( n + 1 ) b}{2} ] .$$

The nth harmonic may be a wavelet with a frequency higher than a fundamental wave of the signal, wherein n is an integer greater than 1. In some embodiments, the nth harmonic of the resonant frequency f may be a distorted wavelet of fundamental wave output from the resonant circuit 121, which may cause electromagnetic interference to the load. In some embodiments, the nth harmonic of the effective frequency range may be a wavelet of the fundamental wave of the load signals. In some embodiments, a frequency of the nth harmonic represents n times the resonant frequency f. For example, when the resonant frequency is f, a frequency of the third harmonic may be 3f, a frequency of the fifth harmonic may be 5f. In some embodiments, a frequency of the nth harmonic of the effective frequency range may be n times the load frequency, accordingly, when the effective frequency range is [a, b], the nth harmonic frequency range of the effective frequency range may be

$$[ \frac{( n + 1 ) a + ( n - 1 ) b}{2} , \frac{( n - 1 ) a + ( n + 1 ) b}{2} ] .$$

To avoid the electromagnetic interference of the signal and the harmonic to the load, the effect of the load may be reduced by limiting the signal frequency. In some embodiments, the resonant frequency f and the nth harmonic of the resonant frequency f may be adjusted to be outside the effective frequency range [a, b] of the load and the nth harmonic frequency range of the effective frequency range range

$$[ \frac{( n + 1 ) a + ( n - 1 ) b}{2} , \frac{( n - 1 ) a + ( n + 1 ) b}{2} ] ] .$$

For the signal of the load, the signal of the resonant circuit 121 and the signal of the harmonic may be outside a useful bandwidth of the load signal, the load may filter the interference by means of digital or analog filtering and signal processing, which may avoid affecting the operation of the load in the frequency range [a, b] and the nth harmonic frequency range of the effective frequency range

$$[ \frac{( n + 1 ) a + ( n - 1 ) b}{2} , \frac{( n - 1 ) a + ( n + 1 ) b}{2} ] ] ,$$

so as to further reduce the EMI of the power supply apparatus 100 to the load and improve the operating quality of the load.

In some embodiments, by arranging a shielded shell sleeved on the outside in the power supply apparatus 100, an electromagnetic environment inside the shell may be separated from an environment outside the shell, which may further affect the load caused by the EMI interference.

In some embodiments, the resonant circuit 121 may further include a switch (e.g., Q1 and Q2 shown in FIG. 4) and a driver (e.g., U1 shown in FIG. 4), an electrical energy input end of the driver may be connected with the power supply circuit 110, the driver may receive the drive signal and control the switch, the switch may be connected with the power supply circuit 110, and the switch may receive the first power supply.

The driver U1 may be an integrated circuit that drives the switch for state switching (e.g., on and off states). In some embodiments, a drive end PWM of the driver U1 may receive the drive signal, and the driver U1 may control the switch according to the drive signal. For example, the drive end PWM of the driver U1 may receive the pulse width modulation (PWM) signal, and the driver U1 may output high and low levels and control on and off of the switch according to a PWM ratio of the PWM signal. More descriptions of the specific implementation manner of the drive signal may be found in the related descriptions of the following drive circuit, which may not be repeated here.

It should be noted that, in some embodiments, the adjustment of the resonant frequency of the resonant circuit 121 may need to consider switching damage and interference of the switch for the user. Taking the switching damage as an example, the lower the resonant frequency is, the lower the first switching frequency may be and the lower the switching loss may be, which may increase the efficiency of the resonant circuit 121. Taking the interference of the switch as an example, if the resonant frequency is outside a user hearing frequency range, a sound signal generated when the switch is switched on and off may avoid being captured by the user's hearing, which may reduce the interference to the user.

In some embodiments, the electrical input end of the driver U1 may be connected with the power supply circuit 110 and used to receive the second power supply provided by the power supply circuit 110. More descriptions of the specific implementation manner of the second power supply may be found in the above related descriptions of the power supply circuit 110, which may not be repeated here. In some embodiments, a drive output pin UGATE and a drive output pin LGATE of the drive U1 may be connected with a corresponding control end of the switch respectively, which may be used to output a control signal. In some embodiments, an enable end EN/PG of the driver U1 may be used to receive an enable signal, and the driver U1 may control the driver U1 on and off according to the enable signal. In some embodiments, a mode selection end BOOT of the driver U1 may be connected with a phase end PHASE by a bootstrap capacitor C41, which may provide a trigger voltage for the field effect transistor Q1. In some embodiments, the phase end PHASE of the driver U1 may be connected with a source electrode of a field effect transistor Q1 and a drain electrode of a field effect transistor Q2, to provide a backflow path. In some embodiments, a ground end GND of the drive U1 may be grounded.

The switch may be a component that controls the circuit on or off. In some embodiments, a control end of the switch may receive the control signal (e.g., the input drive signal), and the switch may switch its own on or off according to the control signal. Taking FIG. 4 as an example, the switch may include a field effect transistor Q1 and a field effect transistor Q2. A gate of the field effect transistor Q2 may be connected with a drive output pin LGATE of the driver U1, a drain electrode of the field effect transistor Q1 may be connected with the power supply circuit 110, the source electrode of the field effect transistor Q1 may be connected with the drain electrode of the field effect transistor Q2, and a source electrode of the field effect transistor Q2 may be grounded. A connection point of the source electrode of the field effect transistor Q1 and the drain electrode of the field effect transistor Q2 may be connected with the phase end PHASE of the drive U1 and the inductor, respectively. In some embodiments, the field effect transistor Q1 and the field effect transistor Q2 may be conducted alternately according to the drive signal, to provide the electric energy for the inductor and the resonant capacitor. More description of the specific control process of the switch may be found in the following related descriptions of the operating process of the resonant circuit 121, which may not be repeated here.

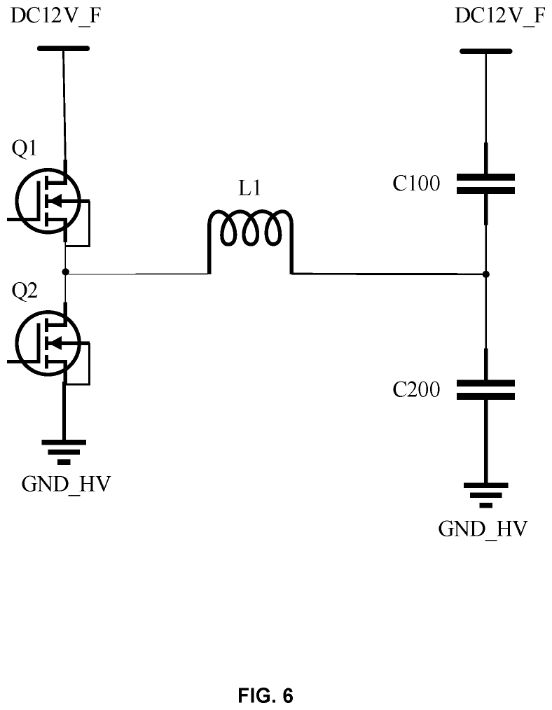

To describe the operating process of the resonant circuit 121 clearer, the resonant circuit 121 as shown in FIG. 4 may be simplified as the resonant circuit 121 as shown in FIG. 6.

FIG. 6 is a schematic diagram illustrating a simplified structure of a resonant circuit 121 according to some embodiments of the present disclosure.

In some embodiments, the resonant capacitor bank may include a first resonant capacitor subset (an equivalent resonant capacitor C100 as shown in FIG. 6) and a second resonant capacitor subset (an equivalent resonant capacitor C200 as shown in FIG. 6), one end of the first resonant capacitor subset may be connected with one end of the second resonant capacitor subset, another end of the first resonant capacitor subset may be used to receive the biasing voltage, and another end of the second resonant capacitor subset may be grounded.

The first resonant capacitor subset and the second resonant capacitor subset may be equivalent capacitor banks of the resonant circuit 121, such as equivalent capacitor banks C100 and C200 as shown in FIG. 6. In some embodiments, the first resonant capacitor subset may include capacitors C17-C21 and C25-C27, and the second resonant capacitor subset may include capacitors C1, C15-C16, and C22-C24 as shown in FIG. 4. In some embodiments, by adjusting one or more of the count, parameter, connection manner of the capacitors C1 and C15-C27, the parameters of the first resonant capacitor subset and the second resonant capacitor subset may be adjusted, so that the resonant frequency of the resonant circuit 121 may be adjusted quickly.

In some embodiments, the first resonant capacitor subset and the second resonant capacitor subset may be symmetrically arranged with a connection end of the first resonant capacitor subset and the second resonant capacitor subset as a center. In some embodiments, the connection end of the first resonant capacitor subset and the second resonant capacitor subset may include one or more connection points for electrical connection. As shown in FIG. 4, the connection end of the first resonant capacitor subset and the second resonant capacitor subset may include connection points for electrical connection of the capacitor C1, the capacitor C22, the capacitor C23, the capacitor C24, and the capacitor C15 in the first resonant capacitor subset, and the capacitor C17, the capacitor C20, the capacitor C21, the capacitor C27, and the capacitor C25 in the second resonant capacitor subset.

In some embodiments, the symmetrical arrangement may include one or more symmetrical manners such as a symmetrical position and a symmetrical capacitance value of the first resonant capacitor subset and the second resonant capacitor subset, and a symmetrical current flowing through the first resonant capacitor subset and the second resonant capacitor subset. That is to say, the equivalent resonant capacitors C100 and C200 may be symmetrically arranged with a connection end of the equivalent resonant capacitors C100 and C200 as a center, so that the current of the first resonant capacitor subset and the second resonant capacitor subset may be symmetrical, to reduce the ripple in the output of the power supply apparatus 100.

In some embodiments, a difference in capacitance between the first resonant capacitor subset and the second resonant capacitor subset may be less than or equal to 5% of either capacitance of the first resonant capacitor subset or the second resonant capacitor subset. For example, if a capacitance value of the first resonant capacitor subset is X and a capacitance value of the second resonant capacitor subset is Y, |X−Y|≤5% X or |X−Y|≤5% Y.

In some embodiments, the field effect transistor Q1, the field effect transistor Q2, and the inductor L1 as shown in FIG. 6 may correspond to the field effect transistor Q1, the field effect transistor Q2, and the inductor L1 as shown in FIG. 4, respectively. More description of the specific connection manner of the above components as shown in FIG. 6 may be found in the related descriptions of the connection manner as shown in FIG. 4.

It should be noted that, without the biasing voltage, the resonant circuit 121 may control a current direction in the resonant circuit 121 to switch by switching the state of the switch (e.g., on and off), so that part of alternating component may be transmitted and diffused through a circuit board, and the output of the power supply apparatus 100 may include the ripple, which may generate the EMI to the load.

To reduce the ripple generated in the resonant circuit 121, in some embodiments, one end of the first resonant capacitor subset may be arranged with the biasing voltage. In this way, when the state of the switch switches, the biasing voltage may be matched with the first power supply, to make a symmetrical current of the first resonant capacitor subset and the second resonant capacitor subset in the resonant circuit 121 and reduce the ripple in the output of the power supply apparatus 100, which may avoid the interference to the load.

For example, as shown in FIG. 6, if the first power supply and the biasing voltage provide a 12V DC voltage, the field effect transistors Q1 and Q2 may from two current loops, and one of the two current loops may include the field effect transistor Q1, the inductor L1, the equivalent resonant capacitor C200 (i.e., the second resonant capacitor subset), another one of the two current loops may include the field effect transistor Q2, the inductor L1, the equivalent resonant capacitor C100 (i.e., the first resonant capacitor subset). When the field effect transistor Q1 is turned on and the field effect transistor Q2 is turned off, a voltage at both ends of the equivalent resonant capacitor C200 may be +6V, a voltage at both ends of the inductor L1 may be +6V, and the current may flow from the field effect transistor Q1 to the inductor L1. When the field effect transistor Q1 is turned off and the field effect transistor Q2 is turned on, a voltage at both ends of the equivalent resonant capacitor C100 may be +6V, a voltage at both ends of the inductor L1 may be −6V, and the current may flow from the inductor L1 to the field effect transistor Q2. That is to say, voltages of the both ends of the equivalent resonant capacitors C100 and C200 may be a superposition of +6V DC biasing voltage and the voltage of the resonant signal, and a voltage of the both ends of the inductor L1 may be a ±6V square wave drive voltage.

Therefore, when a duty ratio of the drive signal PWM is 50%, 50% of the current may flow to the position of 12V, 50% of the current may flow to the position of 0V, to make a symmetrical current of the equivalent resonant capacitor C100 and the equivalent resonant capacitor C200 and reduce the ripple in the output of the resonant circuit 121.

In some embodiments, the processing unit 120 may further include a processing circuit, an input end of the processing circuit may be connected with an output end of the booster circuit, and a forward voltage output end of the processing circuit may provide the forward power supply, and a backward voltage output end of the processing circuit may provide the negative power supply.

In some embodiments, the processing circuit may include: functional circuits such as a rectifier circuit, a filter circuit, a regulator circuit, or one or more combinations thereof. In some embodiments, the processing circuit may include a filter and regulator circuit, for example, the processing circuit may include an LDO circuit. In some embodiments, the processing circuit may include a rectifier and filter circuit, for example, the processing circuit may include a DC half-wave rectifier voltage multiplication circuit. It should be noted that the processing circuit and the processing unit 120 may be different circuit components, the processing unit 120 may convert the first power supply into the forward power supply and the negative power supply, and the processing circuit may perform electrical processing such as rectification, filtration, and voltage stabilization on high-voltage electrical energy output by the booster circuit.

In some embodiments, the processing circuit may process the output of the booster circuit to obtain the forward power supply and the negative power supply, and a polarity of the forward power supply may be opposite to a polarity of negative power supply. The following takes the rectifier and filter circuit and the filter and regulator circuit as an example to describe the specific implementation manner of the processing circuit.

In some embodiments, as shown in FIG. 4, when the booster circuit is the resonant circuit 121, the processing circuit may include a rectifier and filter circuit.

In some embodiments, a difference between a voltage of the biasing voltage and a voltage of the first power supply may be within a present voltage range. In some embodiments, a capacitance value of the first resonant capacitor subset and a parameter of the first resonant capacitor subset may be similar or the same, and the field effect transistor Q1 and the field effect transistor Q2 may be similar or the same. In this way, the voltage of the biasing voltage may be similar to or the same as the voltage of the first power supply, which makes a voltage at both ends of the first resonant capacitor subset similar to or the same as a voltage at both ends of the second resonant capacitor subset and further reduces the ripple in the output of the resonant circuit 121.

In some embodiments, the processing unit 120 may further include a rectifier and filter circuit, an input end of the rectifier and filter circuit may be connected with the output end of the resonant circuit 121, and the rectifier and filter circuit may be arranged with the resonant circuit 121 as a center, a first output end of the rectifier and filter circuit may be used to provide the forward power supply, a second output end of the rectifier and filter circuit may be used to provide the negative power supply; a symmetrical degree between the forward power supply and the negative power supply may be within a preset symmetrical range.

The rectifier and filter circuit may be a circuit that converts the AC electrical energy into the DC electrical energy. In some embodiments, the rectifier and filter circuit may include a first rectifier and filter circuit subcircuit 122 and a second rectifier and filter circuit subcircuit 123, and the first rectifier and filter circuit subcircuit 122 and the second rectifier and filter circuit subcircuit 123 may be arranged with the resonant circuit 121 as a center. An input end of the first rectifier and filter circuit subcircuit 122 and an input end of the second rectifier and filter circuit subcircuit 123 may receive the resonant signals, respectively, and process the resonant signals by using a same rectifier and filter processing manner, to provide the first rectifier and filter circuit subcircuit 122 and the second rectifier and filter circuit subcircuit 123 with relatively high symmetry. In some embodiments, the first rectifier and filter circuit subcircuit 122 and the second rectifier and filter circuit subcircuit 123 may be DC half-wave rectifier voltage multiplication circuits.

In some embodiments, the preset symmetrical range may represent a symmetrical degree between the forward power supply and the negative power supply. For example, the smaller the preset symmetrical range is, the more symmetrical the forward power supply and the negative power supply may be. The larger the preset symmetrical range is, the smaller the symmetrical degree between the forward power supply and the negative power supply may be. In some embodiments, by adjusting the structure of the rectifier and filter circuit, the preset symmetrical range may be adjusted. In some embodiments, the preset symmetrical range may be [0.99, 1). Preferably, the present symmetrical range may be [0.999, 1).

The following provides a structure of the rectifier and filter circuit to describe the symmetry.

In some embodiments, as shown in FIG. 4, the first rectifier and filter subcircuit may include capacitors C3, C6-C8, and diodes D3-D4. One end of the capacitor C3 may be connected with the output end of the resonant circuit 121, and another end of the capacitor C3 may be connected with a connection end of the capacitor C6 and capacitor C7. The capacitor C6-C8 may be connected in parallel, one end of the capacitor C6 may be grounded, one end of the capacitor C8 may be grounded, and a connection point of the capacitor C7 and the capacitor C8 may be used to provide the forward power supply. An anode of the diode D3 may be connected with a cathode of the diode D4, and a cathode of the diode D3 may be connected with the connection point of the capacitor C7 and the capacitor C8. The cathode of the diode D4 may be connected with a connection point of the capacitor C6 and the capacitor C7, and an anode of the diode D4 may be grounded.

In some embodiments, as shown in FIG. 4, the second rectifier and filter subcircuit may include capacitors C2, C9-C11, and diodes D1-D2. One end of the capacitor C2 may be connected with the output end of the resonant circuit 121, and another end of the capacitor C2 may be connected with a connection point of the capacitor C9 and the capacitor C10. The capacitor C9-C11 may be connected in parallel, one end of the capacitor C11 may be grounded, and a connection point of the capacitor C10 and the capacitor C11 may be used to provide the negative power supply. An anode of the diode D1 may be at least connected with the connection point of the capacitor C9 and the capacitor C10, and the cathode of the diode D1 may be grounded. A cathode of the diode D2 may be connected with an anode of the diode D1, and the anode of the diode D2 may be connected with a connection point of the capacitor C10 and the capacitor C11.

In this way, the structure of the first rectifier and filter subcircuit and the structure of the second rectifier and filter subcircuit may be arranged with the resonant circuit 121 as a center and may rectify and filter the resonant signal output by the resonant circuit 121 symmetrically.

The following takes the first rectifier and filter subcircuit and the second rectifier and filter subcircuit as an example to describe a rectification and filtering process.

If the resonant signal of the resonant signal is a single frequency sine wave, amplitude may be Vₘ. For the first rectifier and filter subcircuit, in a negative half cycle of the resonant signal, the diode D4 may be turned on, the diode D3 may be turned off, and the resonant signal may charge the capacitor C3 to Vₘ-V_{D} through the diode D4, in a positive half cycle of the resonant signal, the diode D4 may be turn off, the diode D3 may be turned on, and the resonant signal may charge the capacitor C8 through the capacitor C3 and the diode D3. Since a voltage at both ends of the capacitor C3 may be Vₘ-V_{D}, plus Vₘ of the resonant capacitor and a tube voltage drop of the diode D3, the capacitor may be charged to 2(Vₘ-V_{D}), since a direction of the diode, a direction of the voltage at both ends of the capacitor C11 may be the same as a direction of the resonant signal, and the forward power supply may be +HV.

For the second rectifier and filter subcircuit, in the positive half cycle of the resonant signal, the diode D1 may be turned on, the diode D2 may be turned off, and the resonant signal may charge the capacitor C2 to Vₘ-V_{D} (V_{D} may be a tube voltage drop of the diode) through the diode D1, in the negative half cycle of the resonant signal, the diode D1 may be turned off, the diode D2 may be turned on, and the resonant signal may charge the capacitor C11 through the capacitor C2 and the diode D2. Since a voltage at both ends of the capacitor C2 is Vₘ-V_{D} (the voltage of the capacitor cannot change suddenly), plus Vₘ of the resonant capacitor C and a tube voltage drop of the diode D2, the capacitor C11 may be charged to 2(Vₘ-V_{D}), since directions of the diode D1-D2, a direction of the voltage at both ends of the capacitor C11 may be opposite to the direction of the resonant signal, and the negative power supply may be −HV.

In some embodiments, a plurality of charging combination of the capacitors and the diodes may be set in the rectifier and filter subcircuit for double voltage charging. Taking the first rectifier and filter subcircuit as an example, the plurality of charging combination composed of the capacitors C6-C7 and the diodes D3-D4 may be set, so that the capacitor C8 may be charged to 2n(Vₘ-V_{D}). Accordingly, the forward power supply may be +nHV.

It should be noted that the load capacitors C11 and C8 may be charged to 2(Vₘ-V_{D}) in one or more cycles. After completing the charging, if the load consumes the power, the voltages of the capacitors C11 and C8 may be reduced, then the charging process may be repeated, which makes the output voltage keeps at a certain value. In some embodiments, the capacitors C11 and C8 may be composed of the plurality of capacitors, the capacitors C11 and C8 may be used to store the energy and smooth the filtering, which may play the role of rectification and filtering with the diodes D1-D4 and convert the AC signal output by the resonant circuit 121 into the DC power supply.

In this way, by the symmetrical structure of the rectifier and filter circuit, the resonant signals may be processed by using the same rectification and filtering manner, respectively, to improve the symmetry between the forward power supply and the negative power supply and avoid generating a second harmonics to cause the EMI to the load.

In some embodiments, the first rectifier and filter circuit may further include an inductor L3, one end of the inductor L3 may be connected with a connection point of the capacitor C7 and the capacitor C8, and another end of the inductor L3 may be used to output a forward target power supply +HV_F. Accordingly, the second rectifier and filter circuit may further include an inductor L4, one end of the inductor L1 may be connected with a connection point of the capacitor C10 and the capacitor C11, and another end of inductor L4 may be used to output a negative target power supply −HV_F. The inductor L3 and the inductor L4 may be used as magnetic beads to filter the output, so that the forward target power supply +HV_F and the negative target power supply −HV_F may be provided to reduce the EMI interference of the power supply apparatus to the load.

In some embodiments, the current of the forward power supply and the current of the negative power supply may be within a preset current range.

In some embodiments, the preset current range may indicate that the current of the forward supply and the current of the negative power supply are stable. It should be noted that, since the electrical energy conversion is performed by using the resonant circuit 121, the current of the forward supply and the current of the negative power supply may be stable, and a charging time may be controlled by using a constant current charging manner. For example, when the ultrasound device generates a 128-channel 4.0 MHz full-wave signal pulse with a duration of 240 ns, a voltage on the load capacitor with an equivalent capacitance of 35 uF may drop by 1V. According to an equation for charging and discharging the capacitor (i.e., an Equation (2)), a time for charging the load capacitance may be calculated. The equation for charging and discharging the capacitor may be:

$$I = C \times \frac{d V}{dt} \qquad ( 2 )$$

wherein I denotes a current output by the power supply apparatus, C denotes a capacitance value of the load capacitor, V denotes a voltage at both ends of the capacitor, and t denotes a time for charging and discharging the power. In this way, if the voltage drop of the load capacitance r is 1V, the power supply apparatus 100 needs to output 0.5 A current to charge 70 us for recharging. The charging time may be increased by reducing the current output by the power supply apparatus 100, or the charging time may be reduced by increasing the current output by the power supply apparatus 100.

In some embodiments, the current of the forward power supply and the current of the negative power supply may be adjusted according to the load need for the current. In some embodiments, the current of the forward power supply and the current of the negative power supply may be controlled by the load current and the load capacitance.

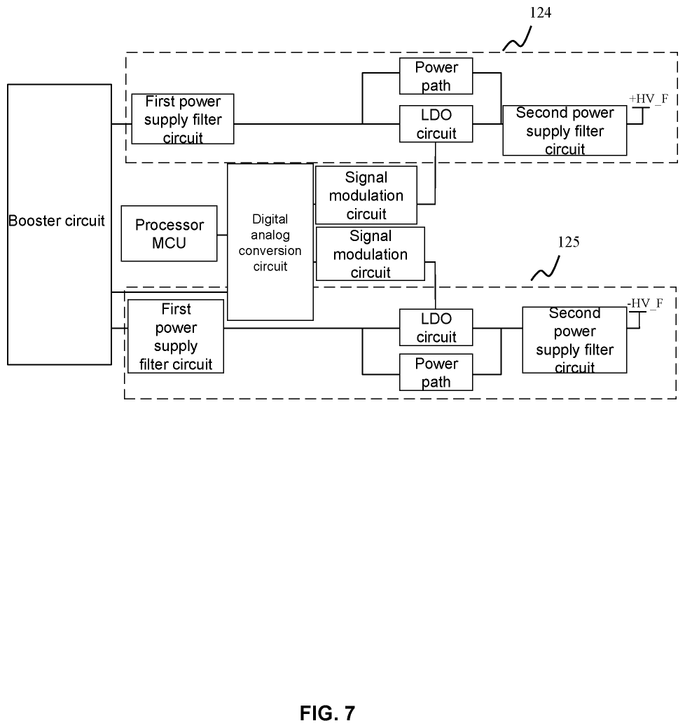

FIG. 7 is a schematic diagram illustrating a structure of a processing unit 120 according to some embodiments of the present disclosure.

In some embodiments, as shown in FIG. 7, the processing unit 120 may further include a filter and regulator circuit, an input end of the filter and regulator circuit may be connected with an output end of the booster circuit, the filter and regulator circuit may be symmetrically arranged with the booster circuit as a center, a first output end of the filter and regulator circuit may be used to provide the forward power supply, and a second output end of the filter and regulator circuit may be used to provide the negative power supply; the symmetrical degree between the forward power supply and the negative power supply may be within the preset symmetrical range.

The filter and regulator circuit may be a circuit for removing signal impurities. In some embodiments, the filter and regulator circuit may include a first filter and regulator subcircuit 124 and a second filter and regulator subcircuit 125, and the first filter and regulator subcircuit 124 and the second filter and regulator subcircuit 125 may be symmetrically arranged with the booster circuit as a center. An input end of the first filter and regulator subcircuit 124 and an input end of the second filter and regulator subcircuit 125 may process the output of the booster circuit by using a same filtering and regulating process manner, to output the forward power supply and the negative power supply with a relatively high symmetry. In some embodiments, the first filter and regulator subcircuit 124 and the second filter and regulator subcircuit 125 may be LDO circuits. The following provides the exemplary structure of the filter and regulator circuit to describe the symmetry.

In some embodiments, as shown in FIG. 7, the first filter and regulator subcircuit 124 may include a first power supply filter circuit, an LDO circuit, a power path, and a second power supply filter circuit. The first power supply filter circuit, the LDO circuit, and the second power supply filter circuit may be connected in sequence, an input end of the first power supply filter circuit may be connected with an output end of the booster circuit, the second power supply filter circuit may be used to output the forward power supply, and the power path may be connected with the LDO circuit in parallel.

In some embodiments, the power supply filter circuit may be a circuit for filtering signal noise, such as an LC filter circuit or an RC filter circuit. In some embodiments, the LDO circuit may reduce the switching noise. Since the LDO circuit usually has a relatively small input voltage range and a relatively large energy consumption, for increasing the input voltage range of the LDO and reducing the energy consumption, in some embodiments, the LDO of the first filter and regulator subcircuit 124 may use floating ground design. In some embodiments, the power path may be a circuit with shunt function. In some embodiments, the power path may reduce current of the LDO circuit by being connected in parallel with the LDO circuit. The parallel power path in the load, current in the LDO circuit may be reduced and the power consumption of the LDO circuit may be reduced. In some embodiments, the power path may be power devices such as MOSFET.

Taking the filter and regulator circuit as shown in FIG. 4 as an example, the specific implementation manner of the first filter and regulator subcircuit 124 may be described in detail. In some embodiments, the first filter and regulator subcircuit 124 may reduce the noise output by the booster circuit by using the first power supply filter circuit, and reduce the switching noise by using the LDO circuit. The most current output by the first power supply filter circuit may be transmitted by the power path to reduce the LDO power consumption. In some embodiments, the LDO circuit may reduce the noise in the electrical energy output by the LDO circuit by the second power supply filter circuit to output the forward power supply. The specific implementation manner of the second filter and regulator subcircuit 125 may be the same as the performing manner of the first filter and regulator subcircuit 124, which may not be repeated here.

In some embodiments, the processing unit 120 may include a digital-to-analog conversion circuit, an input end of the digital-to-analog conversion circuit may be connected with the processor, and the first output end of the digital-to-analog conversion circuit may be connected with the drive end of the booster circuit. In some embodiments, the digital-to-analog conversion circuit may convert the output of the processor into a voltage signal, and transmit the voltage signal to the booster circuit through the first output end of the digital-to-analog conversion circuit to control a voltage output by the booster circuit.

In some embodiments, the processing unit 120 may further include a signal modulation circuit. An input end of the signal modulation circuit may be connected with a second output end of the digital-to-analog conversion circuit, and an output end of the signal modulation circuit may be connected with the LDO circuit. In some embodiments, the digital-to-analog conversion circuit may convert the output of the processor into the voltage signal, process the voltage signal by the signal modulation circuit, and transmit the processed voltage signal to the LDO signal, to control the LDO circuit to filter and regulate the electric energy.

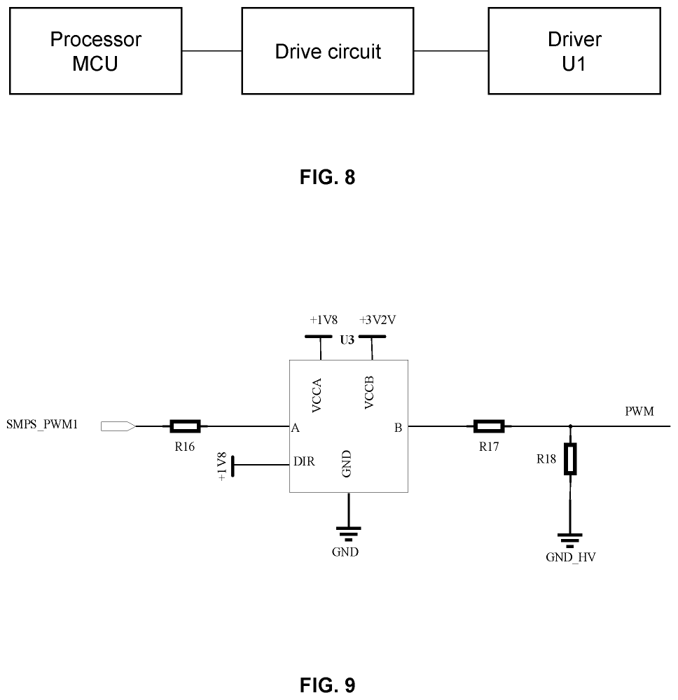

FIG. 8 is a schematic diagram illustrating a structure of a driving circuit according to some embodiments of the present disclosure.

In some embodiments, the power supply apparatus 100 may further include a drive circuit, an input end of the drive circuit may be connected with the processor (MCU), an output end of the drive circuit may be connected with a drive end of the driver U1, and the drive circuit may transmit the instruction according to the processor (MCU) to output the drive signal to the driver U1.

FIG. 9 is a schematic diagram illustrating a circuit structure of a driving circuit according to some embodiments of the present disclosure.

The drive circuit may be a circuit for transmitting the instruction, for example, the instruction (e.g., an initial drive signal) sent by the processor (MCU) may be transmitted to the driver U1. In some embodiments, as shown in FIG. 8, when a level of the initial drive signal output by the processor (MCU) is different from a desired level input from the driver U1, the drive circuit may include a voltage buffer U3, an input end of the voltage buffer U3 may be connected with the processor (MCU), which may receive the initial drive signal of the processor (MCU), an output end of the voltage buffer U3 may be connected with the driver U1, which may transmit the drive signal to the driver U1. The voltage buffer U3 may be an integrated circuit for converting level. In some embodiments, the voltage buffer U3 may adjust a level of the initial drive signal to avoid the driver U1 being unable to process signals with inappropriate level.

In some embodiments, an output end B of the voltage buffer U3 may be connected with the driver U1 through R17, a connection of the resistor R17 and the driver U1 may be connected with one end of the resistor R18, another end of the resistor R18 may be grounded. The resistors R17-R18 may be used for overcurrent protection. In some embodiments, an electrical input end VCCA of the voltage buffer may receive a third power supply, an electrical input end VCCB of the voltage buffer U3 may receive a fourth power supply, a voltage of the third power supply and a voltage of the fourth power supply may be same or different. In some embodiments, a drive end DIR of the voltage buffer U3 may receive the third power supply, so that an input end A of the voltage buffer U3 may receive the signal, and an output end B of the voltage buffer U3 may transmit the signal. The drive end DIR of the voltage buffer U3 may be used to control a flow direction of the signal. In some embodiments, a ground end GND of the voltage buffer U3 may be grounded.

In some embodiments, the processor may transmit the initial drive signal to the driver U1 to control a drive frequency of the driver U1. In some embodiments, the processor may transmit an enable signal to the driver U1 to control the driver U1 on and off. More descriptions of the specific implementation manner of the enable signal may be found in the related descriptions of the switching circuit, which may not be repeated here.

The drive signal may be a signal with variable parameters, such as the initial drive signal sent by the processor (MCU) and the drive signal sent by the voltage buffer U3. In some embodiments, the drive signal may include a carrier signal, a pulse signal, a sine signal, and one or more combinations thereof. For example, the initial drive signal and the drive signal may be pulse width modulation signals. In some embodiments, inherent properties of the drive signal may be used to adjust a first switching frequency of the switch, for example, the drive signal may adjust the working state of the resonant circuit 121.

In some embodiments, by controlling the frequency of the drive signal, the frequency of the control signal output by the driver may be adjusted. In some embodiments, a difference between the frequency of the drive signal and the resonant frequency f may be within a preset range. In some embodiments, by adjusting the frequency of the drive signal, the control signal output by the driver and the drive signal may be close to the resonant frequency f. Taking the driving signal as the pulse width modulation signal as an example, when the pulse width modulation signal and the control signal are close to the resonant frequency of the resonant circuit 121, the resonant circuit 121 may resonate, and the current flowing through the switch (e.g., Q1 and Q2 as shown in FIG. 4) when turned on and off may be 0, so that the loss of the switch may be reduced and the efficiency of the power supply apparatus 100 may be improved.

It should be noted that, during the practical application, the power supply apparatus 100 that provides the power for the load may be one or more. When there are multiple power supply apparatuses 100, to avoid excessive ripple caused by the superposition of drive signals, in some embodiments, the processor (MCU) may control a phase difference between the drive signals, so as to cancel the EMI interference caused by the ripple.

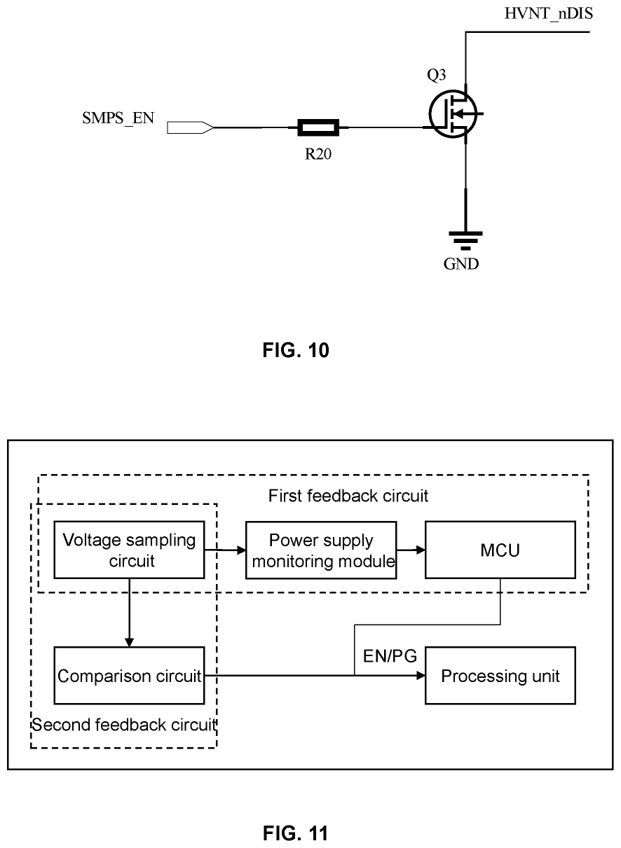

FIG. 10 is a schematic diagram illustrating a structure of a switching circuit according to some embodiments of the present disclosure.

In some embodiments, as shown in FIG. 10, the power supply apparatus 100 may further include a switching circuit, an input end of the switching circuit may be connected with the processor (MCU), which may receive the initial enable signal. An output end of the switching circuit may be connected with an enable end EN/PG of the driver U1, and an output end of the switching circuit may be used to transmit the enable signal. Accordingly, the enable end EN/PG of the driver U1 may be used to receive the enable signal and switch the driver U1 according to the enable signal.

The switching circuit may be a circuit for transmitting the switching instruction. In some embodiments, the switching circuit may drive the switch by using the initial enable signal sent by the processor (MCU) and output the enable signal to the controller U1 by controlling the on and off state of the switch. In this way, the processor (MCU) may realize the function of remotely turning on or off the drive U1, which can reduce the loss of the power supply apparatus 100 effectively and the EMI of the switching circuit to load.

The enable signal may be a signal whose level changes, such as the initial enable signal sent by the processor (MCU) and the enable signal sent by the switching circuit. In some embodiments, the enable signal may include one or more signals such as a square wave signal, a pulse signal, a sine signal, etc. In some embodiments, the enable signal may include a level signal, the apparatus may be controlled to switch the corresponding state by using a high level signal and low level signal. For example, when the enable signal is the high level signal, the driver U1 may be turned on, when the enable signal is the low level signal, the driver U1 may be turned off.

The following provides an exemplary switching circuit to describe a specific implementation manner of remote switch control.

In some embodiments, as shown in FIG. 10, the switching circuit may include a field effect transistor Q3 and a resistor R11, the gate of the field effect transistor Q3 may be connected with the processor (MCU) through the resistor R11, and the drain of the field effect transistor Q3 may be connected with the enable end EN/PG of the controller U1, and may also be connected with the second power supply through the resistor R3, and the source of the field effect transistor Q3 may be grounded. When an initial enable signal SMPS_EN sent by the processor (MCU) is at a high level, the field effect transistor Q3 is turned on, which makes the drain of field effect transistor Q3 directly grounded, and the output signal HVNT_nDIS is low level. When the initial enable signal SMPS_EN sent by the processor (MCU) is at a high level, the field effect transistor Q3 is turned off, under the action of the resistor R3 and the second power supply, a voltage of the drain of the field effect transistor Q3 may rise, and the output enable signal HVNT_nDIS may be at a high level.

FIG. 11 is a schematic diagram illustrating a structure of a feedback circuit according to some embodiments of the present disclosure.

In some embodiments, the power supply apparatus 100 may further include a feedback circuit used to adjust the working state of the processing unit 120 according to the forward power supply and the negative power supply. In some embodiments, the feedback circuit may include a voltage sampling circuit for sampling the output voltages of the forward power supply and the negative power supply to obtain a first sampling voltage and a second sampling voltage. In some embodiments, the feedback circuit may transmit the first sampling voltage and the second sampling voltage to the processor through the power supply monitoring module, so that the processor may transmit the first enable signal to an enable end of the controller U1. A voltage sampling circuit, a power supply monitoring module, and a processor may form a first feedback circuit. In some embodiments, the feedback circuit may compare the first sampling voltage and the second sampling voltage with a reference voltage by setting the comparison circuit, so as to obtain the second enable signal and the third enable signal to be transmitted to the enable end of the controller U1. The voltage sampling circuit and the comparison circuit may form a second feedback circuit. In some embodiments, the first enable signal, the second enable signal, and the third enable signal may be used to adjust the output of the processing unit 120.

When the first feedback circuit and the second feedback circuit coexist, the processing unit 120 may simultaneously receive a first enable signal, a second enable signal, and a third enable signal. In some embodiments, the processing unit 120 may control the working state of the resonant circuit 121 according to the first enable signal, the second enable signal, and the third enable signal. In some embodiments, the processing unit 120 may determine a target enable signal based on the received first enable signal, the second enable signal, and the third enable signal, so as to adjust the output based on the target enable signal.

In some embodiments, the processing unit 120 may process the first enable signal, the second enable signal, and the third enable signal according to an “and” logic. When the first enable signal, the second enable signal, and the third enable signal are all high level, the target enable signal may be the high level, and when any of the first enable signal, the second enable signal, or the third enable signal is low level, the target enable signal may be the low level. Correspondingly, when the target enable signal is the high level, the driver U1 may be turned on, and when the target enable signal is the low level, the driver U1 may be turned off, so that the working state of the resonant circuit 121 may be controlled by regulating the driver U1.

In some embodiments, the use of multiple feedback mechanisms (e.g., when the first feedback circuit and the second feedback circuit coexist) may effectively avoid the inaccuracy of the output power supply caused by a single error.

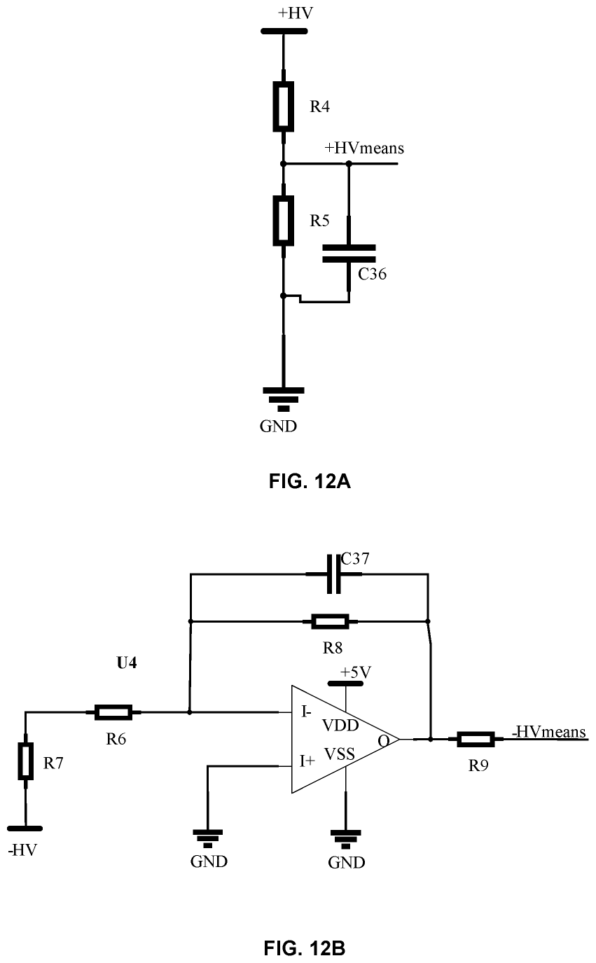

FIGS. 12A and 12B are schematic diagrams illustrating a structure of a voltage sampling circuit according to some embodiments of the present disclosure.

In some embodiments, the voltage sampling circuit may be divided into the forward voltage sampling circuit and the backward voltage sampling circuit, which are used to sample the forward voltage sampling circuit and the backward voltage sampling circuit respectively.

FIG. 12A is a forward voltage sampling circuit. The input of the forward voltage sampling circuit may be a connection point of capacitor C7 and capacitor C8 in the circuit shown in FIG. 4, that is, an output end of the forward power supply. In some embodiments, the forward power supply +HV may be grounded by the resistors R4 and R5, and capacitor C36 may be connected in parallel with both ends of resistor R5, and a connection point of resistance R4 and R5 may be used to provide a first sampling voltage +HVmeans. The resistors R4 and R5 may be used to divide the voltage of the forward power supply, so as to obtain a first sampling voltage +HVmeans with a relatively low voltage value. In some embodiments, a ratio of the resistance values of R4 and R5 may be set to adjust a range of the first sampling voltage.

FIG. 12B is a backward voltage sampling circuit. The input of the forward voltage sampling circuit may be a connection point of capacitor C10 and capacitor C11 in the circuit as shown in FIG. 4, that is, an output end of the negative power supply. In some embodiments, a negative power supply −HV may be connected with a backward input end of the operational amplifier U4 by the resistors R6 and R7, and an output end of the operational amplifier U4 may provide a second sampling voltage −HVmeans by the resistor R9. Meanwhile, the output end of the operational amplifier U4 may be connected with the backward input end of the operational amplifier U4 through the resistor R8, and the capacitor C37 may be connected in parallel with both ends of the resistor R8. The forward input end of the operational amplifier U4 may be grounded, a VDD port may be connected with a fifth power supply, and a VSS port may be grounded. The operational amplifier U4 and the resistors R6, R7, and R8 may form a negative proportional operational amplifier circuit, which converts the negative power supply −HV into a positive voltage for sampling, thereby obtaining the second sampling voltage −HVmeans. In some embodiments, the fifth power supply may be provided by a fifth power supply circuit similar to the second power supply circuit, and the fifth power supply may be a 5V DC power supply.

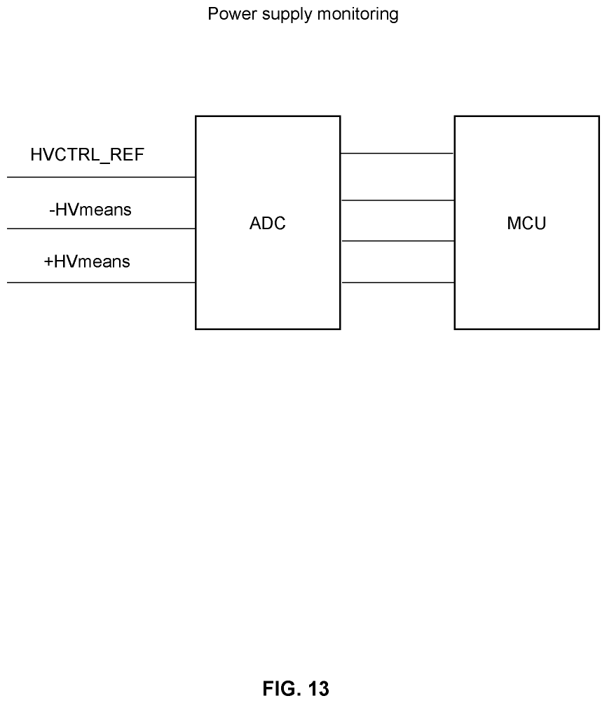

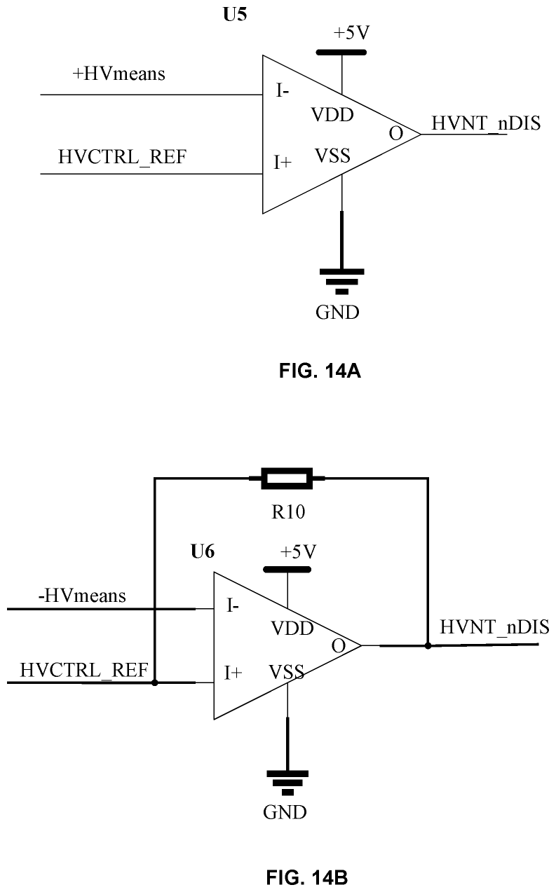

In some embodiments, the first sampling voltage +HVmeans and the second sampling voltage −HVmeans may generate an enable signal for adjusting the output of the processing unit 120 through the first feedback circuit or the second feedback circuit. More descriptions of the specific implementation manner of the first feedback circuit and the second feedback circuit may be found in FIG. 14A, FIG. 14B, FIG. 13, and the related descriptions, which may not be repeated here.

FIG. 13 is a schematic diagram illustrating a structure of a power supply monitoring module according to some embodiments of the present disclosure.

In some embodiments, the first feedback circuit may be configured to receive and output the forward power supply, the negative power supply, and a reference voltage to the processor (MCU), so that the processor (MCU) may transmit a first enable signal to the processing unit 120. The first enable signal may be configured to adjust the output of the processing unit 120.

In some embodiments, the first feedback circuit may include a power supply monitoring module. A first sample voltage and a second sample voltage may be transmitted to the processor (MCU) through the power supply monitoring module, so that the processor (MCU) may transmit the first enable signal. In some embodiments, the power supply monitoring module may include an analog-to-digital converter ADC, and the analog-to-digital converter ADC may include at least three signal receiving channels. The signal receiving channels may be configured to receive the first sample voltage +Hvmeans, the second sample voltage −Hvmeans, and the reference voltage HVCTRL_REF (V_{REF}) that are processed by signal processing. The analog-to-digital converter ADC may convert the voltage signal into digital signal, and the digital signal may be transmitted to the processor (MCU), so that the voltage of the forward power supply and the voltage of the negative power supply may be monitored in real-time.

In some embodiments, when the processing unit 120 is connected with the power supply, the driver U1 may start to work. The processor (MCU) may transmit instructions to the driver U1 through a driver circuit. After the driver U1 receives the driver signal consistent with the resonant frequency f, the resonant circuit 121 and the rectifier and filter circuit may start to work by controlling the on and off states of the switches Q1 and Q2, so that the voltages output by the forward power supply +HV and the negative power supply −HV may continuously increase. In some embodiments, a voltage sampling circuit may sample the forward power supply +HV and the negative power supply −HV to obtain the first sample voltage +HVmeans and the second sample voltage −HVmeans. The first sample voltage +Hvmeans, the second sample voltage −Hvmeans, and the reference voltage HVCTRL_REF may be converted into the digital signal (e.g., the voltage) through the analog-to-digital converter ADC, and the digital signal may be transmitted to the processor (MCU).

The processor (MCU) may compare the voltage transmitted by the analog-to-digital converter ADC with a threshold. In some embodiments, when the voltage transmitted by the analog-to-digital converter ADC is greater than a first threshold V_{TH1}, the processor (MCU) may output the first enable signal with a low electrical level to the processing unit 120. The first enable signal HVNT_nDIS₁ may be connected with the enable end EN/PG of the driver U1, the driver U1 may be disabled, the switches Q1 and Q2 may be turned off, and the resonant circuit 121 may stop working, so that the voltages output by the forward power supply +HV and the negative power supply −HV may not continue to increase. For example, combined with the switching circuit shown in FIG. 10, when the voltage transmitted by the analog-to-digital converter ADC is greater than the first threshold V_{TH1}, an initial enable signal SMPS_EN of the processor (MCU) may be a high electrical level, the field effect transistor Q3 may be turned on, so that the drain electrode of the field effect transistor Q3 may be directly grounded, and the output first enable signal HVNT_nDIS₁ may be the low electrical level.

Due to load consumption or the like, the voltages output by the forward power supply +HV and the negative power supply −HV may decrease. In some embodiments, when the voltage transmitted by the analog-to-digital converter ADC is less than a second threshold V_{TH2}, the processor (MCU) may output the first enable signal with a high electrical level to the processing unit 120. The driver U1 may be enabled, the switches Q1 and Q2 may be turned on, and the resonant circuit 121 may start to work, so that the voltages output by the forward power supply +HV and the negative power supply −HV may continuously increase. For example, combined with the switching circuit shown in FIG. 10, when the voltage transmitted by the analog-to-digital converter ADC is less than the first threshold V_{TH2}, an initial enable signal SMPS_EN of the processor (MCU) may be a low electrical level, the field effect transistor Q3 may be turned off, so that the output first enable signal HVNT_nDIS1 may be the low electrical level.

When the voltages output by the forward power supply +HV and the negative power supply −HV continue to increase to a certain value, the operations mentioned above may be repeated, so that the forward power supply +HV and the negative power supply −HV may keep dynamic balance and remain within a certain range.

In some embodiments, in order to prevent frequent enabling of the driver U1, the first threshold V_{TH1} may not be equal to the second threshold V_{TH2}, and the second threshold V_{TH2} may be less than the first threshold V_{TH1}. In some embodiments, the first threshold V_{TH1}, and the second threshold V_{TH2} may be set based on the voltage of the high voltage power supply required by the load and the sensitivity to the power supply voltage.

FIGS. 14A and 14B are schematic diagrams illustrating a circuit structure of a comparison circuit according to some embodiments of the present disclosure.

In some embodiments, the second feedback circuit may include a first comparison circuit and a second comparison circuit. The first comparison circuit may include a first comparator U5. The first comparator U5 may output a first comparison result based on the forward power supply and the reference voltage. The second comparison circuit may include a second comparator U6. The second comparator U6 may output a second comparison result based on the negative power supply and the reference voltage.

In some embodiments, the second feedback circuit may transmit a second enable signal and a third enable signal to the processing unit 120 based on the comparison result between the sample voltage of the forward power supply and the reference voltage output by the first comparator U5 and the comparison result between the sample voltage of the negative power supply and the reference voltage output by the second comparator U6 (i.e., the first comparison result and the second comparison result). The second enable signal and the third enable signal may be configured to adjust the output of the processing unit 120.

In some embodiments, when the processing unit 120 is connected with the power supply, the driver U1 may start to work. The processor (MCU) may transmit instructions to the driver U1 through the driver circuit. After the driver U1 receives the driver signal consistent with the resonant frequency f, the resonant circuit 121 and the rectifier and filter circuit may start to work by controlling the on and off states of the switches Q1 and Q2, so that the voltages output by the forward power supply +HV and the negative power supply −HV may continuously increase. In some embodiments, a voltage sampling circuit may sample the forward power supply +HV and the negative power supply −HV to obtain the first sample voltage +HVmeans and the second sample voltage −HVmeans. The output of the processing unit 120 may be adjusted by generating the enable signal through the first comparison circuit and the second comparison circuit.

Refer to the first comparison circuit shown in FIG. 14A, a backward input end of the first comparator U5 may be the first sample voltage +Hvmeans, and a forward input end of the first comparator U5 may be the reference voltage HVCTRL_REF. The VDD port may be connected with the sixth power supply, the VSS port may be grounded, and the output end may be the second enable signal HVNT_nDIS2, which is connected with the EN/PG pin of the enable end of the driver U1.

In some embodiments, when the first sample voltage +HVmeans is greater than the reference voltage V_{REF}, the first comparator U5 may output the second enable signal HVNT_nDIS₂ with a low electrical level. The drive output pin UGATE of the driver U1 may be controlled to disconnect from LGATE, the switches Q1 and Q2 may be turned off, and the resonant circuit 121 may stop working, so that the voltages output by the forward power supply +HV and the negative power supply −HV may not continue to increase. Due to load consumption or the like, the voltage output by the forward power supply +HV may decrease. In some embodiments, when the first sample voltage +HVmeans is greater than the reference voltage V_{REF}, the first comparator U5 may output the second enable signal HVNT_nDIS₂ with a high electrical level. The driver U1 may be enabled and the resonant circuit 121 may start to work, so that the voltage output by the forward power supply +HV may increase to a certain range of a threshold, and finally may reach a balance value to obtain a required output voltage.

In some embodiments, a relationship between the forward power supply and the reference voltage may be denoted as Equation (3):

$$+ H V = + HVmean s ( ( R 4 + R 5 ) / R 5 ) = {V}_{R E F} ( ( R 4 + R 5 ) / R 5 ) \qquad ( 3 )$$

wherein +HV refers to the voltage of the forward power supply, +Hvmeans refers to the first sample voltage, V_{REF} refers to the reference voltage, R4 refers to the resistance value of the resistor R4, and R5 refers to the resistance value of the resistor R5. In some embodiments, assuming that R4=100K Ω, R5=2.7K Ω, and the reference voltage V_{REF} is 1.8V, so that the voltage of the forward power supply is 68.47V.

Referring to the second comparison circuit shown in FIG. 14B, a backward input end of the second comparator U6 may be the second sample voltage −HVmeans, and a forward input end of the second comparator U6 may be the reference voltage HVCTRL_REF. The VDD port may be connected with the sixth power supply, the VSS port may be grounded, and the output end may be the third enable signal HVNT_nDIS₃, which is connected with the EN/PG pin of the enable end of the driver U1. Since the second sample voltage −HVmeans is a positive voltage, the working principle of the second comparator U6 may be similar to the working principle of the first comparator U5. The third enable signal may be output by comparing the second sample voltage −HVmeans and the reference voltage V_{REF} to control the on and off states of the resonant circuit 121, and a required output voltage may be obtained.

In some embodiments, the output ends of the first comparison circuit and the second comparison circuit may be connected together to be connected with the EN/PG pin of the enable end of the driver U1. In some embodiments, when the first feedback circuit and the second feedback circuit coexist, a target enable signal HVNT_nDIS may be generated based on the first enable signal HVNT_nDIS1, the second enable signal HVNT_nDIS2, and the third enable signal HVNT_nDIS3 through an “AND” logic of a consolidation circuit. The target enable signal HVNT_nDIS may be transmitted to the enable end EN/PG of the driver U1 to control a working state of the resonant circuit 121.

In some embodiments, the second comparison circuit may include a hysteresis resistor R10. The hysteresis resistor R10 may be arranged between the forward input end of the second comparator U6 and the output end of the second comparator U6 to adjust a threshold voltage of the comparator. Since the voltage of the load storage capacitor may decrease with the consumption of the load, and the charging current is constant, the capacitor may be charged and discharged intermittently, and the voltage may be the integral of the charging current over time, therefore, the control loop may have a 90° phase delay. In order to improve the stability of the loop and reduce the frequency of recharging, a loop compensation circuit may also be arranged in the second comparison circuit, which is realized by the hysteresis resistor R10. Details may refer to descriptions of FIG. 16, which is not repeated herein.

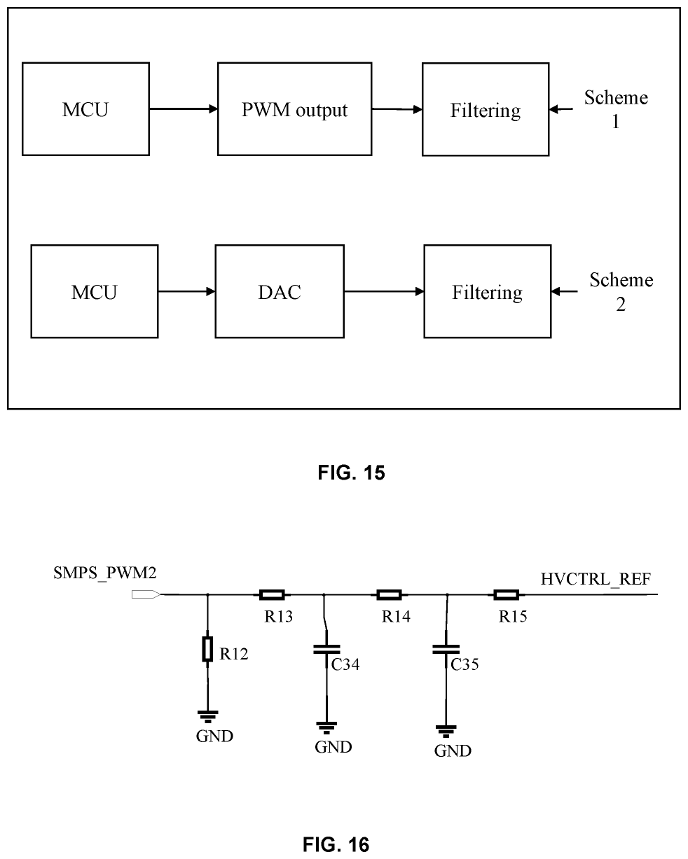

FIG. 15 is a schematic diagram illustrating a generation manner of a reference voltage according to some embodiments of the present disclosure.

In some embodiments, the power supply apparatus may include a reference voltage circuit. The reference voltage circuit may be configured to provide an adjustable voltage to the first feedback circuit (i.e., the power supply monitoring module) and the second feedback circuit (including the first comparison circuit and the second comparison circuit), so that the output voltage may be adjusted.

The reference voltage circuit may include a plurality of implementing manners. For example, referring to the scheme 1 shown in FIG. 15, the processor (MCU) may control and output a second drive signal SMPS_PWM2 with a certain frequency, the second drive signal may be a PWM waveform, and then a DC reference voltage HVCTRL_REF may be obtained after filter processing. Referring to the scheme 2 shown in FIG. 15, the processor (MCU) may control the digital-to-analog converter DAC to output a certain voltage, and then the reference voltage HVCTRL_REF may be obtained after filter processing.

The specific implementation of the reference voltage circuit may mainly consider whether the reference voltage may be adjusted, and the adjustment accuracy of the reference voltage. For the scheme 1, the DC voltage obtained by filter processing may be proportional to the duty cycle of the second drive signal, therefore, the adjustment accuracy of the reference voltage may mainly depend on the frequency of the second drive signal and the adjustable range of the duty cycle. For the scheme 2, the accuracy of adjusting the reference voltage by controlling the output through the digital-to-analog converter DAC may depend on a count of bits of the digital-to-analog converter DAC.

FIG. 16 is a schematic diagram illustrating a circuit structure of a reference voltage circuit according to some embodiments of the present disclosure. As shown in FIG. 16, the scheme 1 shown in FIG. 15 illustrates the filter circuit after the second drive signal SMPS_PWM2 is controlled and output by the processor (MCU).

In some embodiments, the second drive signal SMPS_PWM2 may be generated by a second drive circuit having a similar structure to the structure of a first drive circuit. The processor (MCU) may be connected with the second drive circuit and send instructions to the second drive circuit. The second drive circuit may generate the second drive signal SMPS_PWM2. In some embodiments, the second drive signal SMPS_PWM2 may obtain the reference voltage HVCTRL_REF through two-stage RC filter processing.

As shown in FIG. 16, one end of SMPS_PWM2 may output the reference voltage V_REF through the resistors R13, R14, and R15 that are connected in series. The connection point between one end of SMPS_PWM2 and the resistor R13 may be grounded through the resistor R12, the connection point between the resistors R13 and R14 may be grounded through the capacitor C34, and the connection point between the resistors R14 and R15 may be grounded through the capacitor C35.

In some implementations, the second drive signal SMPS_PWM2 with different duty cycles or frequencies may be obtained by the processor (MCU) outputting different instructions, so that the reference voltage HVCTRL_REF may be adjusted.

As shown in FIG. 4, FIG. 14B, and FIG. 16, the hysteresis resistor R10 may be arranged between the output end of the second comparator U6 and the forward input end of the second comparator U6. The output end of the second comparator U6 may be connected with the enable end of the driver U1, and the enable end of the driver U1 may also be connected with the second power supply through the resistor R3. Therefore, the hysteresis resistor R10 may be pulled up to the third power supply (e.g., 7V power supply) through the resistor R3. Since the forward input end of the second comparator U6 is connected with the reference voltage V_REF shown in FIG. 15, the hysteresis resistor R10 and the resistors R13, R14, and R15 may form a voltage divide circuit, and the generated voltage may be superimposed on the reference voltage V_{REF}, so that a certain feedback hysteresis may be generated at the output end HVNT_nDIS2 of the second comparator U6, thereby improving the stability of the feedback loop. The hysteresis resistor R10 may ensure that the charging time interval required by the load during no pulse transmission may not be too frequent, which effectively reduces the frequent on or off of the driver U1, the switch Q1, and the switch Q2, thereby reducing switching losses and improving power efficiency.

In some embodiments, the voltage superimposed on the reference voltage V_{REF} through voltage division in the voltage divide circuit may be calculated by Equation (4):

$${V}_{div} = \frac{R 13 + R 14 + R 15}{R 3 + R 10 + R 13 + R 14 + R 15} \qquad ( 4 )$$

The voltage fluctuation fed back to the output end may be calculated by Equation (5):

$${V}_{hys} = {V}_{d i v} ( ( R 4 + R 5 ) / R 5 ) \qquad ( 5 )$$

wherein V_{div} refers to the voltage superimposed on the reference voltage V_{REF} through voltage division, V_{hys} refers to the voltage fluctuation fed back to the output end, R3, R4, R5, R10, R13, R14, and R15 refer to resistances of the resistors R3, R4, R5, R10, R13, R14, and R15.

When the processing unit starts to work, the reference voltage V_{REF} may be greater than the first sample voltage +HVmeans, the driver U1 may be enabled, and the voltage of the forward power supply may increase gradually. When the voltage increases to

$$( {V}_{R E F} + {V}_{div} ) ( \frac{R 4 + R 5}{R 5} ) ,$$

the second comparator U6 may output a low electrical level, and V_{div}=0V. When the output voltage decreases to

$$( {V}_{R E F} ) ( \frac{R 4 + R 5}{R 5} ) ,$$

the second comparator U6 may output a high electrical level to cause the driver U1 to be enabled. The driver U1 may control the switches Q1 and Q2 to be turned on, and the resonant circuit 121 may start to work. A fixed power supply fluctuation V_hys may appear at the output voltage end, which reduces the frequent on or off of the driver, reduces the switching loss, improves the power supply efficiency, and achieves phase compensation for the loop stability.

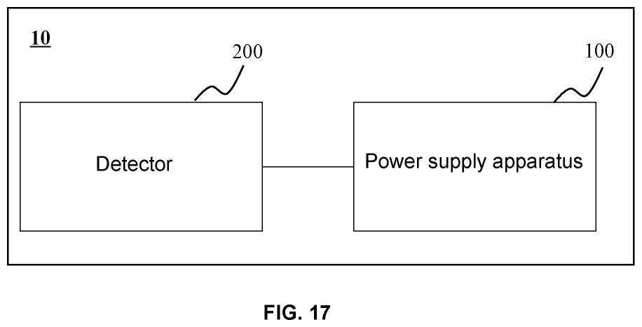

FIG. 17 is a schematic diagram illustrating a structure of a medical apparatus according to some embodiments of the present disclosure.

In some embodiments, as shown in FIG. 17, the embodiments of the present disclosure may provide a medical apparatus 10. The medical apparatus 10 may include a power supply apparatus 100 and a detector 200. In some embodiments, the power supply apparatus 100 may provide the forward power supply and the negative power supply to the detector 200 to facilitate the working of the detector 200. Specific implementation of the power supply 100 may refer to descriptions of FIG. 1 to FIG. 16, which is not repeated herein. In some embodiments, the detector 200 may be a detection apparatus that uses positive and negative high-voltage electrical energy to collect signals and particles, such as an energy spectrum detector, a radiation detector, an ultrasonic detector, or the like. Specific implementation of the detector 200 may refer to descriptions of the load, which is not repeated herein.

It should be noted that the power supply apparatus 100 may also be used in production scenarios in which positive and negative high-voltage electrical energy is used to generate signals such as pulses, lasers, or the like, or in detection scenarios in which positive and negative high-voltage electrical energy is used to collect signals, particles, or the like. The present disclosure may not limit the specific application scenario of the power supply apparatus 100.

The present disclosure may provide a power supply apparatus (i.e., a high-voltage power supply) configured to provide high-voltage signal and a corresponding medical apparatus. Following may describe the specific implementation manners.

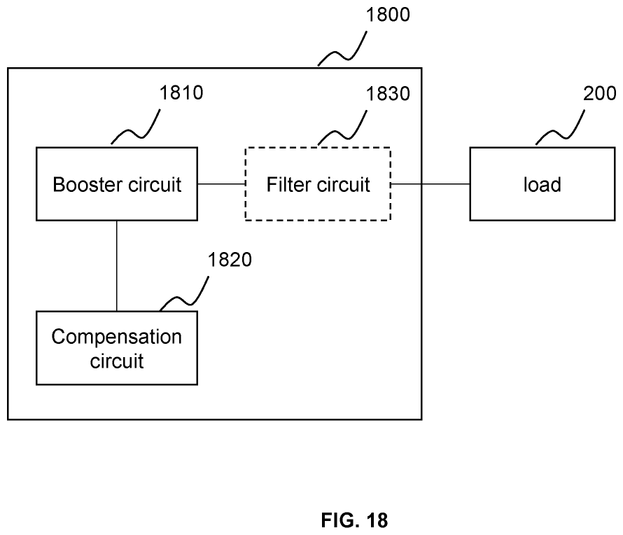

FIG. 18 is a schematic diagram illustrating a structure of a medical imaging apparatus according to some embodiments of the present disclosure. In some embodiments, the medical imaging apparatus may include a high-voltage power supply 1800 and a load 200. An input end of the high-voltage power supply 1800 may be connected with an input power supply, and an output end of the high-voltage power supply 1800 may be connected with the load 200. In some embodiments, the high-voltage power supply may be a power supply apparatus, which converts the power supply signal into the high-voltage signal, and transmits the high-voltage signal to the load 200 to provide energy to the load 200.

In some embodiments, the load 200 may be an electronic apparatus that operates with the high voltage signal. For example, the load 200 may be a detector that requires a high-voltage field for collection (e.g., an energy spectrum detector in the medical field, etc.), or a separator that requires a high-voltage field to separate substances (e.g., a gas recoverer, a coke oven gas purifier, etc.). In some embodiments, the load 200 may be an electronic apparatus in the medical imaging field. Further, the load 200 may be an energy spectrum detector in the medical imaging field.

In some embodiments, the input power supply may provide the electrical energy to the high-voltage power supply 1800. For example, the input power supply may be an AC power supply to provide an AC power supply signal to the high-voltage power supply 1800. As another example, the input power supply may also be a DC power supply to provide a DC power supply signal to the high-voltage power supply 1800. Electronic energy properties of the input power supply may be selected based on the structure of the high-voltage power supply 1800.

In some embodiments, the high-voltage power supply 1800 may include a booster circuit 1810 and a compensation circuit 1820. An input end of the booster circuit 1810 may be connected with the input power supply, and an output end of the booster circuit 1810 may be configured to output electrical energy. An input end of the compensation circuit 1820 may be at least connected with the input end of the booster circuit 1810, and an output end of the compensation circuit 1820 may be at least connected with a reference end of the booster circuit 1810. The compensation circuit 1820 may be configured to adjust the output electrical power of the booster circuit 1810 based on a voltage of the input power supply.

In some embodiments, the booster circuit 1810 may be a circuit structure improving the voltage. The voltage of the output electrical power of the booster circuit 1810 may be greater than the voltage of the input power. For example, the booster circuit 1810 may convert the power supply signal of the input power supply into the high-voltage signal, and the voltage of the high-voltage signal may be greater than the voltage of the power supply signal. In some embodiments, the voltage of the high-voltage signal may be 1˜10 kv. In some embodiments, the booster circuit 1810 may be a combination of one or more of a boost booster circuit, a Sepic booster circuit, a voltage doubler circuit, an LC resonant circuit, or a flyback circuit. In some embodiments, the booster circuit 1810 may also convert properties of electrical energy. For example, the flyback circuit may convert an input DC signal into an AC signal, and then convert the AC signal into the output DC signal. Taking the booster circuit 1810 as the flyback circuit as an example, following may describe the working principle of the booster circuit 1810 may be described in detail.

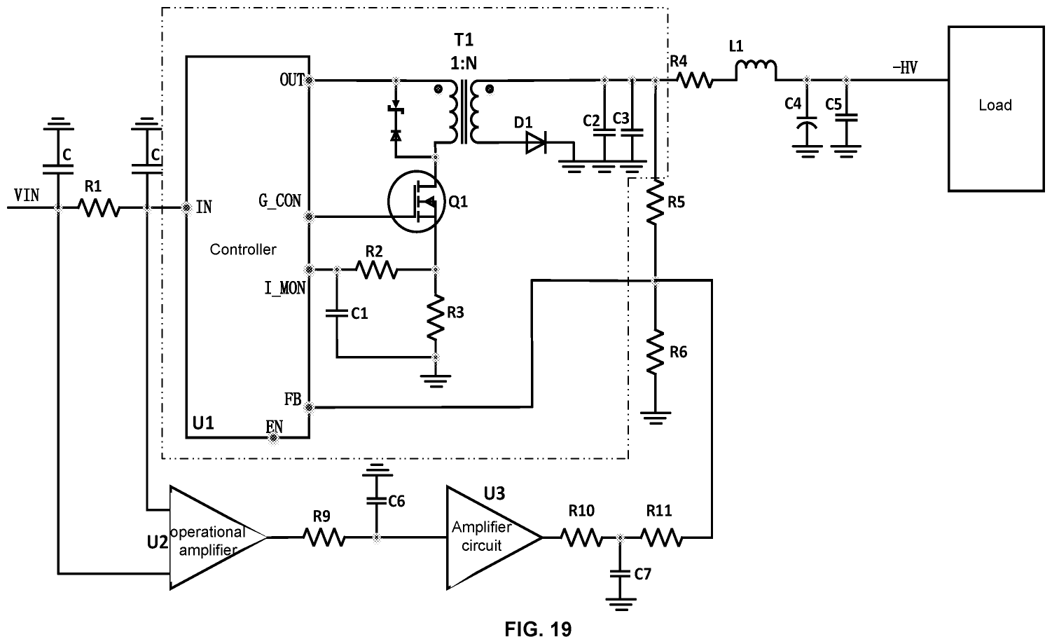

FIG. 19 is a schematic diagram illustrating a circuit structure of a high-voltage power supply according to some embodiments of the present disclosure. In some embodiments, the booster circuit 1810 may include a controller U1, a transformer T1, a switch Q1, a diode D1, a filter capacitor C2, and a filter capacitor C3. An input end IN of the controller U1 may be at least connected with the input power supply, a power output end OUT of the controller U1 may be connected with a primary side input end of the transformer T1, and a drive output end G_CON of the controller U1 may be connected with a control end of the switch Q1. An input end of the switch Q1 may be connected with a primary side output end of the transformer T1, and an output end of the switch Q1 may be grounded. A secondary side input end of the transformer T1 may be connected with an anode of the diode D1, and a cathode of the diode D1 may be grounded. A secondary side output end of the transformer T1 may be connected with first ends of the filter capacitors C2 and C3, and second ends of the filter capacitors C2 and C3 may be grounded. The first ends of the filter capacitors C2 and C3 may be connected with the filter circuit.

The controller U1 may be an integrated circuit that changes the properties of electrical energy. In some embodiments, the input end IN of the controller U1 may receive a power supply signal from the input power supply, and increase the voltage of the power supply signal to output a first voltage signal through the power output end. The voltage of the first voltage signal may be greater than the voltage of the power supply signal. In some embodiments, the drive end of the controller U1 may output the drive signal to control the switch Q1 to be turned on and turned off periodically, so that the primary side magnetic field of the transformer T1 may change, thereby realizing the change from a DC signal to an AC signal. In some embodiments, the drive signal may be a pulse width modulated signal. The controller U1 may adjust the voltage of the output electrical power of the booster circuit 1810 by adjusting the duty cycle of the pulse width modulation signal. In some embodiments, the electrical energy may be an energy form that uses electricity to perform work in various forms. Current, voltage, power, or the like may be parameters of the electrical energy. Following parameters of the electrical energy may be simplified. For example, the voltage of the output electrical power of the booster circuit 1810 may be simplified as the voltage output by the booster circuit 1810.

In some embodiments, a reference end FB of the controller U1 may receive the reference voltage from the compensation circuit 1820, compare the reference voltage with a preset voltage range, and adjust the duty cycle of the PWM signal based on the comparison result, so as to adjust the voltage output by the booster circuit 1810. For example, when the reference voltage is greater than the preset voltage range, the voltage currently output by the booster circuit 1810 may be relatively large, and the controller U1 may reduce the output voltage. When the reference voltage is less than the preset voltage range, the voltage currently output by the booster circuit 1810 may be relatively small, and the controller U1 may increase the output voltage. In some embodiments, adjusting the reference voltage may be equivalent to adjusting an equivalent resistance to ground of the reference voltage, so as to adjust a feedback proportional coefficient of the resistor and control the voltage output by the booster circuit 1810. For the process of adjusting the voltage output by the booster circuit 1810, refer to the following descriptions of the compensation circuit 1820, which is not repeated herein.

In some embodiments, the enable end EN of the controller U1 may receive an enable signal from an abnormity detection circuit 1840. The controller U1 may control the working state of the booster circuit 1810 based on the enable signal. Details about controlling the working state of the booster circuit 1810 may refer to descriptions of the abnormity detection circuit 1840, which is not repeated herein.

In some embodiments, the booster circuit 1810 may further include the resistor R2, the resistor R3, and the capacitor C1. A current detection pin I_MON of the controller U1 may be connected with the output end of the switch Q1 through the resistor R2. The output end of the switch Q1 may be grounded through the resistor R3. The current detection pin I_MON may also be grounded through the capacitor C1. In some embodiments, the current detection pin I_MON of the controller U1 may be filtered through the capacitor C1, and the resistor R2 and the resistor R3 may detect the current on the switch Q1. The controller U1 may compare the current on the switch Q1 with a preset current threshold, and adjust the output electrical power of the controller U1 based on the comparison result. For example, when the current on the switch Q1 is greater than the preset current threshold, the controller U1 may limit the output electrical power of the booster circuit 1810, thereby achieving overcurrent protection of the load 200.

The transformer T1 may be a device that converts the AC voltage into another AC voltage with the same frequency and a different voltage. In some embodiments, the transformer T1 may convert a first voltage signal into a second voltage signal by switching the turn-on state and the turn-off state of the switch Q1, and the voltage of the second voltage signal may be greater than the voltage of the first voltage signal. For example, when the switch Q1 is turned off, the diode D1 on the secondary side of the transformer T1 may be turned on, so that the transformer T1 may output the second voltage signal and charge the filter capacitors C2 and C3; when the switch Q1 is turned on, the diode D1 on the secondary side of the transformer T1 may be turned off in a reverse direction, so that the filter capacitors C2 and C3 may output the second voltage signal. It should be noted that the on and off states of the switch Q1 may cause an alternating component to be generated in the booster circuit 1810, so that the output electrical power of the booster circuit 1810 may have ripples. Specific filter processing of the ripples may refer to the descriptions of the filter circuit 1830, which is not repeated herein.

In some embodiments, the output electrical power of the booster circuit 1810 may be related to winding parameters of the transformer T1. For example, the current direction of the output electrical power of the booster circuit 1810 may be related to a winding direction of the transformer T1. The windings may be wound along a same direction, and an output voltage direction may be opposite to the voltage direction of the primary side input power (e.g., as shown in FIG. 19, the output electrical power of the booster circuit 1810 is a negative high voltage). As another example, the voltage of the output electrical power of the booster circuit 1810 may be related to a winding turns ratio of the transformer T1.

The compensation circuit 1820 may be a circuit structure that compensates parameters of an apparatus to avoid changes in the output of the apparatus. In some embodiments, the compensation circuit 1820 may control the input power of the reference end of the booster circuit 1810, adjust the equivalent resistance to ground of the reference voltage of the booster circuit 1810, thereby adjusting the feedback proportional coefficient to ensure the output electrical power of the booster circuit 1810 to be stable. For example, when the load current increases, in order to avoid the voltage drop of the output electrical power of the booster circuit 1810, the compensation circuit 1820 may reduce the equivalent resistance to ground of the reference voltage of the booster circuit 1810, and increase the feedback proportional coefficient, thereby increasing the voltage output by the booster circuit 1810, which is offset with the voltage drop, to keep the stability of the booster circuit 1810. As another example, when the voltage output by the booster circuit 1810 changes, to keep the output electrical power of the booster circuit 1810 to be stable, the compensation circuit 1820 may make corresponding adjustments based on the feedback proportional coefficient and the reference voltage, thereby controlling the output voltage of the booster circuit 1810 to be stable within the preset voltage range.

In some embodiments, the input of the reference end FB of the booster circuit 1810 may include the reference voltage. The equivalent resistance to ground of the reference voltage may correspond to the current of the input power supply, and the equivalent resistance to ground may also correspond to the voltage of the output electrical power of the booster circuit 1810. In some embodiments, the equivalent resistance to ground of the reference voltage may correspond to the adjusted feedback proportional coefficient, and the adjusted feedback proportional coefficient may correspond to the current of the input power supply.

The reference voltage may be a voltage that is fed back based on the output voltage. In some embodiments, when the output voltage of the booster circuit 1810 is stable, the reference voltage may also be within the preset voltage range. In some embodiments, the equivalent resistance to ground may be the equivalent resistance of the reference end FB of the booster circuit 1810 relative to the ground, and the voltage across the equivalent resistance to ground may be the reference voltage. In some embodiments, the voltage divide effect may be adjusted by adjusting the equivalent resistance to ground, so as to adjust the output voltage of the booster circuit 1810 when the reference voltage is stable. The feedback proportional coefficient may be a coefficient to measure the voltage divide effect of the equivalent resistance to ground. The feedback proportional coefficient may be related to the equivalent resistance to ground. In some embodiments, when the reference voltage is stable, adjusting the feedback proportional coefficient may adjust the voltage divide effect, thereby adjusting the output voltage of the booster circuit 1810.

In some embodiments, the output electrical power of the booster circuit 1810 may be controlled by adjusting the equivalent resistance to ground of the reference voltage to adjust the feedback proportional coefficient. For example, to compensate the voltage drop, the equivalent resistance to ground of the reference voltage may be decreased, and the feedback proportional coefficient may be increased, so that the booster circuit 1810 may increase the voltage of the output electrical power.

In some embodiments, the equivalent resistance to ground may correspond to the current of the input power supply. It should be noted that when the booster circuit 1810 works at a static operating point, based on a power conversion equation, if the input voltage and output voltage are constant, the input current may be proportional to the output current. When the booster circuit 1810 works at the static operating point, the booster circuit 1810 may be in a state that internal components do not consume power, and the quiescent current Iq may be zero. That is, the current of the input power supply may be proportional to the load current, and the equivalent resistance to ground may change based on the change of the current of the input power supply, so as to realize compensation before the voltage drops. For example, when the current of the input power supply increases, the load current and the internal loss of the booster circuit 1810 may increase, and the voltage drop may occur. Therefore, the equivalent resistance to ground of the reference voltage may be controlled to decrease, so that the output voltage of the booster circuit 1810 may be increased, which is offset with the voltage drop and keeps the stability of the output electrical power of the booster circuit 1810.

In some embodiments, the power conversion equation may refer to Equation (6):

$$\frac{{V}_{i n}}{{I}_{i n}} \times η = \frac{{V}_{0}}{{I}_{0}} \qquad ( 6 )$$

wherein Vᵢₙ refers to the voltage of the input power supply, Iᵢₙ refers to the current of the input power supply, V₀ refers to the output voltage of the booster circuit 1810, I₀ refers to the output current of the booster circuit 1810, and η refers to a constant that is related to the static operating point of the booster circuit 1810. When the booster circuit 1810 works at the static operating point, the constant η may be stable. If the voltage Vᵢₙ of the input power supply and the output voltage V₀ of the booster circuit 1810 are constant, the current Iᵢₙ of the input power supply may be proportional to the output current Iᵢₙ of the booster circuit 1810.

In some embodiments, the compensation circuit 1820 may include a current acquisition subcircuit and a compensation resistor. An input end of the current acquisition subcircuit may be at least connected with the input end of the booster circuit 1810, an output end of the current acquisition subcircuit may be at least connected with one end of the compensation circuit, and another end of the compensation circuit may be at least connected with the reference end of the booster circuit 1810. The compensation resistor may control the equivalent resistance to ground based on the current of the input power supply.

The current acquisition subcircuit may be a circuit used to collect current. In some embodiments, the current acquisition subcircuit may obtain the current to be collected through the voltage across a resistor. In some embodiments, as shown in FIG. 19, the current acquisition subcircuit may include an operational amplifier U2 and an acquisition resistor R1. The acquisition resistor R1 may be arranged with the input end of the booster circuit 1810. A first input end and a second input end of the operational amplifier U2 may be connected with both ends of the acquisition resistor R1, respectively. An output end of the operational amplifier U2 may be at least connected with the compensation resistor. The voltage of the output electrical power of the operational amplifier U2 may be controlled by the voltage of the acquisition resistor R1, and the voltage of the acquisition resistor R1 may correspond to the current of the input power supply.

The acquisition resistor R1 may be a resistor that the current to be collected flows through. In some embodiments, the acquisition resistor R1 may convert the current of the input power supply into the voltage of the resistor, so that the current acquisition subcircuit may collect the current of the input power supply by collecting the voltage of the resistor R1. For example, when the current of the input power supply flows into the acquisition resistor R1, since the acquisition resistor has impedance, a difference may occur in the potentials across the acquisition resistor R1. The operational amplifier U2 may be a circuit unit that calculates the difference between the input signals, such as a differential operational amplifier. In some embodiments, the operational amplifier U2 may output the current (i.e., the current of the input power supply) of the acquisition resistor R1 based on the potential difference between the first input end and the second input end.

In some embodiments, the both ends of the acquisition resistor R1 may be provided with an energy storage capacitor C, respectively, and may be grounded. The energy storage capacitor C may provide sufficient energy to the booster circuit 1810 when the current of the input power supply is large.

In some embodiments, the current acquisition subcircuit may further include a filter resistor R9 and a filter capacitor C6. The output end of the operational amplifier U2 may be connected with the compensation resistor through the filter resistor R9, and the filter capacitor C6 may be connected with the connection point of the filter resistor R9 and the compensation resistor. In some embodiments, the filter resistor R9 and the filter capacitor C6 may filter out high-frequency noise in the output electrical power of the operational amplifier U2, and improve the accuracy of current acquisition. In some embodiments, the current acquisition subcircuit may only include the operational amplifier U2 and the acquisition resistor R1 to simplify the circuit structure of the compensation circuit 1820.

In some embodiments, the current acquisition subcircuit may further include an amplifier circuit U3. The output end of the operational amplifier U2 may be connected with the compensation resistor through the filter resistor R9 and the amplifier circuit U3. In some embodiments, the amplifier circuit U3 may be used to amplify the voltage output by the operational amplifier U2 for subsequent compensation.

It should be noted that compared with determining the load current by acquiring the current output by the booster circuit 1810, a common mode voltage of the input power supply may be lower by collecting the current of the input power supply to determine the load current, and the circuit structure of the current acquisition subcircuit may be simple.

The compensation resistor may be a resistor that adjusts the voltage based on the input power. In some embodiments, the compensation resistor may adjust the equivalent resistance to ground of the reference end FB of the booster circuit 1810 based on the output current of the current acquisition subcircuit. In some embodiments, as shown in FIG. 19, the compensation resistor may include a first compensation resistor R10 and a second compensation resistor R11. The first compensation resistor R10 and the second compensation resistor R11 may adjust the equivalent resistance to ground of the reference end FB of the controller U1 based on the output power of the amplifier circuit U3. For example, when the current of the input power supply increases, the current output by the amplifier circuit U3 may also increase. The first compensation resistor R10 and the second compensation resistor R11 may adjust the equivalent resistance to ground of the reference end FB of the controller U1 to be smaller based on the increased current, thereby adjusting the feedback proportional coefficient of the booster circuit 1810, and increasing the voltage output by the booster circuit 1810. Specific adjustment process of the equivalent resistance to ground may refer to the descriptions of the following adjustment process, which is not repeated herein. It should be noted that the compensation resistor may not only include resistors, but also include devices with impedance properties, so that adjusting the voltage based on the input power may be realized, and the equivalent resistance to ground of the reference end FB may be adjusted.

In some embodiments, the compensation circuit 1820 may also include a filter capacitor C7. One end of the filter capacitor C7 may be connected with the connection point of the first compensation resistor R10 and the second compensation resistor R11, and the other end of the filter capacitor C7 may be grounded. The filter capacitor C7 may filter the noise included in the output electrical power of the amplifier circuit U3.

In some embodiments, the reference voltage may correspond to the voltage output by the booster circuit 1810. In some embodiments, the reference voltage may be changed based on the change of the voltage output by the booster circuit 1810, and the output electrical power of the booster circuit 1810 may be kept stable. For example, when the voltage output by the booster circuit 1810 increases, the corresponding reference voltage may increase, so that the booster circuit 1810 may reduce the output voltage based on the increased reference voltage, thereby keeping the output electrical power of the booster circuit 1810 to be stable, and making the load of the high-voltage power supply be in a normal working state. For example, when the output electrical power of the high-voltage power supply is stable, the energy spectrum detector in the medical field may accurately collect the required electrons based on the stable high-voltage field strength, thereby improving the imaging quality.

In some embodiments, the compensation circuit 1820 may include a first voltage divider resistor R5 and a second voltage divider resistor R6 in series. An input end of the first voltage divider resistor R5 may be at least connected with the output end of the booster circuit 1810, and a connection point of the first voltage divider resistor R5 and the second voltage divider resistor R6 may be at least connected with the reference end FB of the booster circuit 1810. The first voltage divider resistor R5, the second voltage divider resistor R6, and the compensation resistor may control the reference voltage.

The voltage divider resistor may be a resistor connected with a circuit in series, which may realize the voltage division when the total voltage remains unchanged. In some embodiments, the first voltage divider resistor R5 and the second voltage divider resistor R6 may divide the voltage output by the booster circuit 1810, so that the voltage output by the booster circuit 1810 may be divided into a voltage across the first voltage divider resistor R5 and a voltage across the second voltage divider resistor R6. In some embodiments, the voltage division effect may be determined based on the feedback proportional coefficient, which is related to a resistance ratio of the voltage divider resistor. In some embodiments, the resistance values of the first voltage divider resistor R5, the second voltage divider resistor R6, and the compensation resistor may determine the feedback proportional coefficient, the voltage across the first voltage divider resistor R5, and the voltage across the second voltage divider resistor R6, so that the output voltage of the booster circuit 1810 may be determined. For example, when the feedback proportional coefficient increases, the voltage across the second voltage divider resistor R6 and the voltage across the compensation resistor may decrease, thereby increasing the voltage output by the booster circuit 1810.

In some embodiments, the first voltage divider resistor R5 and the second voltage divider resistor R6 may adjust the reference voltage input by the reference end FB of the controller U1 based on the voltage output by the booster circuit 1810. For example, when the voltage output by the booster circuit 1810 increases, the voltage across the second voltage divider resistor R6 may also increase, so that the reference voltage input by the reference end FB of the control controller U1 may become larger, and the controller U1 may reduce the voltage output by the booster circuit 1810.

In some embodiments, when the booster circuit 1810 works at the static operating point, the compensation circuit 1820 may obtain the current of the input power supply through the current acquisition subcircuit. The equivalent resistance to ground of the booster circuit 1810 may be adjusted by the compensation circuit 1820 to adjust the output voltage of the booster circuit 1810. In some embodiments, the feedback proportional coefficient may be determined based on a ratio of the equivalent resistance to ground to the first voltage divider resistor R5. Since the reference voltage is stable, in order to illustrate the compensation process more intuitively, assuming that the reference voltage remains unchanged, and the compensation process may be performed by adjusting the equivalent resistance to ground of the booster circuit 1810.

For example, as shown in FIG. 19, the compensation circuit 1820 may collect the voltage at both ends of a sample resistor R1 through the operational amplifier U2 to obtain the current Iᵢₙ of the input power supply Vᵢₙ. The compensation circuit 1820 may also filter the high frequency noise through the filter resistor R9 and the filter capacitor C6. The compensation circuit 1820 may be connected with the reference end FB of the controller U1 through the amplifier circuit U3, the first compensation resistor R10, and the second compensation resistor R11. The compensation circuit 1820 may also equivalently adjust the equivalent resistance to ground RFB of the reference end FB, so as to fine-tune the voltage Vₕᵥ output by the booster circuit 1810.

In some embodiments, the adjustment of the equivalent resistance to ground RFB and the voltage Vₕᵥ output by the booster circuit 1810 may refer to Equation (7) and Equation (8):

$$\{ \begin{matrix}RFB = \vert \frac{{V}_{F B}}{\frac{{I}_{i n} \times R 1 \times A 1 \times A 2 - {V}_{F B}}{R 10 + R 11} + \frac{{V}_{F B}}{R 6}} \vert & ( 7 ) \\ {V}_{h v} = {V}_{F B} \times ( 1 + \frac{R 5}{RFB} ) & ( 8 )\end{matrix}$$

wherein RFB refers to the equivalent resistance to ground of the controller U1, V_{FB} refers to the reference voltage, Iᵢₙ refers to the current of the input power supply Vᵢₙ, A1 refers to an amplification ratio of the operational amplifier U2, A2 refers to an amplification ratio of the amplifier circuit U3, and Vₕᵥ refers to the voltage output by the booster circuit 1810. When the load current of the booster circuit 1810 increases, the current Iᵢₙ of the input power supply Vᵢₙ may increase. Due to the loss of the circuit, the output voltage of the booster circuit 1810 may drop. Due to the compensation circuit, the equivalent resistance to ground RFB may decrease, and the voltage output by the booster circuit 1810 may also increase, so as to compensate the dropped voltage. The compensation circuit 1820 may avoid the power drop that occurs when the booster circuit 1810 changes from a no-load state to a full-load state, and improve the stability of the operation of the load 200.

In some embodiments, the first voltage divider resistor R5 may be a megohm resistor, the second voltage divider resistor R6 may be a kiloohm resistor, and the resistance values of the first compensation resistor R10 and the second compensation resistor R11 may range from 100 M Ω to 1 M Ω. In some embodiments, the resistance value of the first voltage divider resistor R5 may be 2 M Ω, and the resistance value of the second voltage divider resistor R6 may be 2 k Ω.

In some embodiments, the working state of the booster circuit 1810 may also be controlled by the compensation circuit 1820. In some embodiments, the working state of the booster circuit 1810 may be controlled by the first voltage divider resistor R5 and the second voltage divider resistor R6. For example, the booster circuit 1810 may work at the static operating point by adjusting the resistance values of the first voltage divider resistor R5 and the second voltage divider resistor R6, so that when the voltage of the input power supply and the voltage output by the booster circuit 1810 are stable, the current of the input power supply may correspond to the current output by the booster circuit 1810 based on the power conversion equation. Specific correspondence between the current of the input power supply and the current output by the booster circuit 1810 may refer to the descriptions of Equation (7), which is not repeated herein.

Since the switch Q1 in the booster circuit 1810 is turned on and turned off, an alternating component may be generated, and the output of the booster circuit 1810 may have ripples. That is, a switching frequency of the switch Q1 may be a main ripple frequency point. In order to remove the ripples in the output of the booster circuit 1810, a filter circuit may be designed based on the switch Q1 in the booster circuit 1810.

In some embodiments, as shown in FIG. 1, the high-voltage power supply 1800 may include a filter circuit 1830. The filter circuit 1830 may be connected with the output end of the booster circuit 1810, and the filter circuit 1830 may be configured to filter out the output electrical energy of the booster circuit 1830 in a second switching frequency.

In some embodiments, the second switching frequency of the booster circuit 1810 may be the frequency that needs to be filtered. In some embodiments, the second switching frequency of the booster circuit 1810 may be a switching frequency of the switch Q1. That is, the second switching frequency of the booster circuit 1810 may be the main ripple frequency point. For example, (0, 20 MHz] may be a frequency range of the ripples. For the output electrical energy of the booster circuit 1810, the signals in the ripple frequency range may be ripples and noises, which need to be filtered out. Therefore, the filter circuit 1830 may filter the output electrical energy of the booster circuit 1810 at the second switching frequency to reduce the ripples.

The filter circuit 1830 may attenuate the signals in the range of the second switching frequency (e.g., frequencies less than 20 MHz) by more than 18000 times. That is, the signals may be attenuated by −60 dB at the second switching frequency, thereby filtering out the ripples.

In some embodiments, the filter circuit 1830 may include a filter resistor (e.g., the resistor R4 shown in FIG. 19), a filter inductor (e.g., the inductor L1 shown in FIG. 19), and a filter capacitor (e.g., the capacitor C5 or C6 shown in FIG. 19) in series, and one end of the filter resistor may be at least connected with the output end of the booster circuit 1810. The frequency range of the filter circuit 1830 filtering the signals may be controlled by the filter resistor, the filter inductor, and the filter capacitor.

In some embodiments, the filter resistor, the filter inductor, and the filter capacitor may form an RLC low-pass filter. A low-pass filter may be an electronic filter device that allows signals below a cutoff frequency to pass, but does not allow signals above the cutoff frequency to pass. The cutoff frequency may be controlled by the filter resistor, the filter inductor, and the filter capacitor. In some embodiments, the cutoff frequency may be set according to the second switching frequency. The frequency range of the signals filtered by the filter circuit 1830 may be controlled by adjusting the filter resistor, the filter inductor, and the filter capacitor.

In some embodiments, an inductance value of the inductor L1 may be in a range of [1 uH, 10 mH]. In some embodiments, the capacitor C4 may be an electrolytic capacitor, and a capacitance value of the capacitor C4 may be in a range of [1800 nF, 10 uF]. In some embodiments, the capacitor C5 may be a ceramic capacitor, and a capacitance value of the capacitor C5 may be in a range of [1 nF, 1800 nF]. In some embodiments, the resistance of the resistor R4 may be in a range of [10Ω, 18000Ω]. In some embodiments, the resistor R4 may be an OR resistor. Parameters of the components mentioned above may be merely for examples, and the embodiments of the present disclosure may not limit the parameters of the components mentioned above.

It should be noted that compared with other filters, when the RLC low-pass filter performs filter processing, the resistance of the filter resistor may be small, and a OR resistor may be used as the filter resistor, so that the internal loss of the components may be smaller when the load current increases, thereby reducing the voltage drop.

The output end of the filter circuit 1830 may be connected with the load 200 to provide a stable high-voltage power with low ripples to the load 200, so that the load of the high-voltage power supply may be in the normal working state. For example, when the ripples in the output electrical energy of the high-voltage power supply is low, an imaging noise floor of an energy spectrum detector in the medical field may be reduced, thereby improving the imaging quality.

In some embodiments, the filter circuit 1830 may include an active filter. One end of the active filter may be at least connected with the output end of the booster circuit, and another end of the active filter may be at least connected with an external power supply. The input end of the active filter may be provided with a high-impedance resistor, and an active electronic device with a low-impedance may act as an isolation, so that an input impedance of the active filter may be close to infinity and an output impedance may be close to zero under an ideal condition, and a good isolation performance may be achieved. In some embodiments, the active filter may also implement functions of the filter circuit 1830, which may be used to filter the output electronic energy of the booster circuit 1810 in the second switching frequency range. The functions of the active filter may be similar to the RLC low-pass filter, which is not repeated herein.

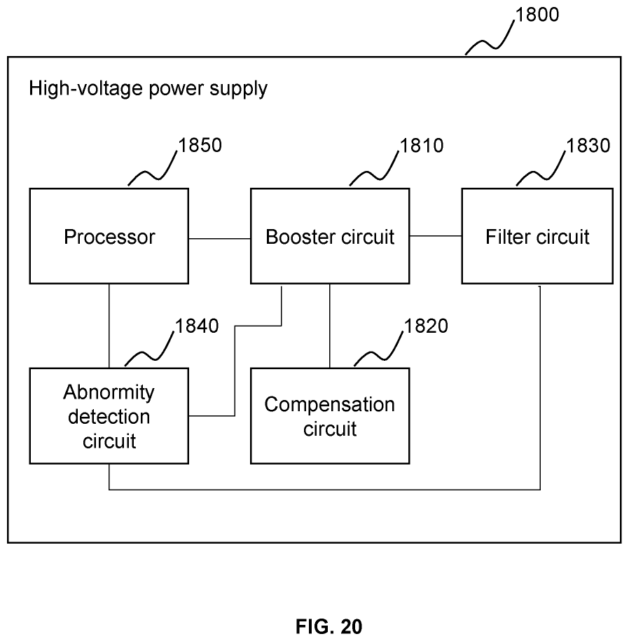

FIG. 20 is a schematic diagram illustrating a structure of a high-voltage power supply according to some embodiments of the present disclosure. In some embodiments, as shown in FIG. 20, the high-voltage power supply may include an abnormity detection circuit 1840 and a processor 1850. A current input end of the abnormity detection circuit 1840 may be at least connected with the input end of the booster circuit 1810, and a voltage input end of the abnormity detection circuit 1840 may be at least connected with an output end of the filter circuit 1830. An input end of the processor 1850 may be connected with a current output end of the abnormity detection circuit 1840 and a voltage output end of the abnormity detection circuit 1840, respectively, and an output end of the processor 1850 may be at least connected with an enable end of the booster circuit 1810. The output electronic energy of the processor 1850 may be controlled by the current of the input power supply and the voltage output by the filter circuit 1830.

The abnormity detection circuit 1840 may be a circuit that detects a working state of the high-voltage power supply. In some embodiments, the abnormity detection circuit 1840 may detect whether the load current and the load voltage are abnormal. In some embodiments, the abnormity detection circuit 1840 may obtain the load current by detecting the current of the input power supply, that is, the current output by the filter circuit 1830. It should be noted that when the booster circuit 1810 works at the static operating point, the load current may correspond to the current of the input power supply. The correspondence between the load current and the input power supply may be similar to the correspondence between the current output by the booster circuit 1810 and the current of the input power supply, which may refer to the descriptions of the current of the input power supply, and may not be repeated herein.

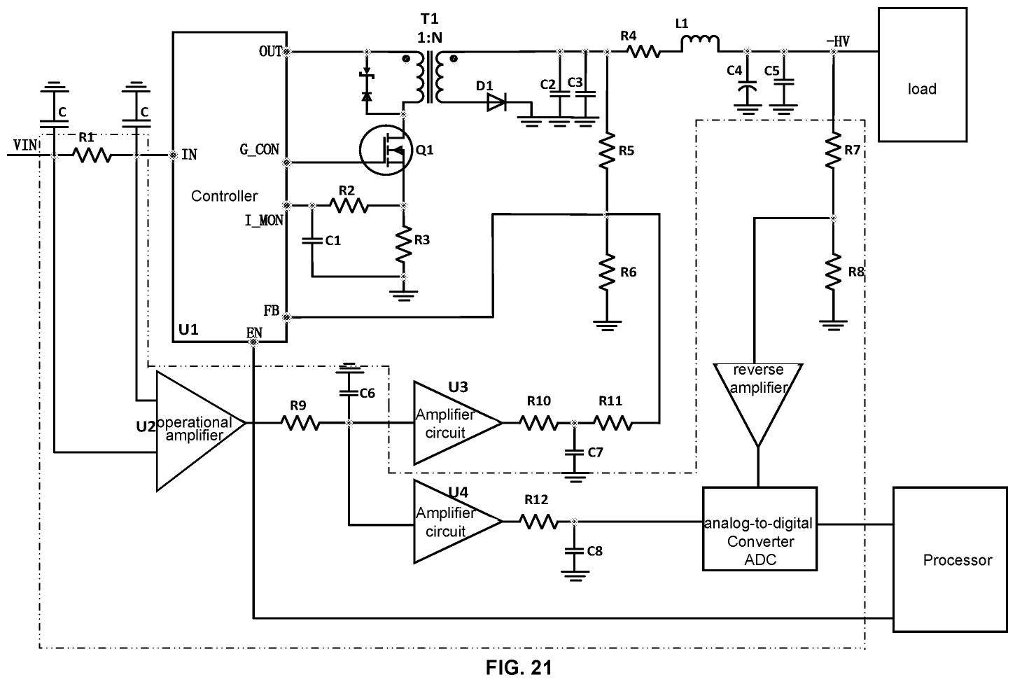

FIG. 21 is a schematic diagram illustrating a circuit structure of a high-voltage power supply according to some embodiments of the present disclosure. In some embodiments, the abnormity detection circuit 1840 may detect whether the current output by the booster circuit 1810 is abnormal by detecting the current of the input power supply. In some embodiments, the abnormity detection circuit 1840 may include a current acquisition subcircuit. An input end of the current acquisition subcircuit may be connected with the input end of the booster circuit 1810, and a current output end of the current acquisition subcircuit may be connected with the processor 1850. The circuit structure of the current acquisition subcircuit in the abnormity detection circuit 1840 may be similar to the circuit structure of the current acquisition subcircuit of the compensation circuit 1820, which is not repeated herein.

In some embodiments, the current acquisition subcircuit in the abnormity detection circuit 1840 and the current acquisition subcircuit in the compensation circuit 1820 may have the same circuit structure. That is, the output end of the current acquisition subcircuit may be connected with the processor 1850 and the compensation resistor at the same time. In some embodiments, as shown in FIG. 21, the abnormity detection circuit 1840 may include an amplifier circuit U4, a filter resistor R12, and a filter capacitor C8 in series. The output end of the current acquisition subcircuit may be connected with the input end of the processor 1850 through the amplifier circuit U4, the filter resistor R12, and the filter capacitor C8 in series. Specific implementation manner of the amplifier circuit U4, the filter resistor R12, and the filter capacitor C8 may refer to the descriptions of the current acquisition subcircuit in the compensation circuit 1820, which is not repeated herein. The following may take FIG. 21 as an example to describe the current acquisition process of the current acquisition subcircuit in detail.

In some embodiments, when the booster circuit 1810 is in the normal working state, a sum of the current output by the booster circuit 1810 and the current when the booster circuit 1810 is in the no-load state may correspond to the current of the input power supply. When the booster circuit 1810 is in the normal working state, the internal components of the booster circuit 1810 may also consume power and a quiescent current Ig may exist in the internal components. In some embodiments, the load current load may be obtained through a current conversion equation based on the voltage Vi of the output electric energy of the current acquisition subcircuit. In some embodiments, the current conversion equation may be denoted as Equation (9):

$${I}_{l o a d} = \frac{\frac{{V}_{i}}{R 1 \times A 1 \times A 3} \times {V}_{i N} \times η}{{V}_{0}} - {I}_{q} \qquad ( 9 )$$

wherein I_{load} refers to the load current, Vi refers to the voltage of the output electric energy of the current acquisition subcircuit, A1 refers to a amplification ratio of the operational amplifier U2, A3 refers to a amplification ratio of the amplifier circuit U4, and Vₕᵥ refers to the voltage of the input power supply, n denotes a constant which is related to the static operating point of the booster circuit 1810, V₀ refers to the voltage output by the filter circuit 1830, and I_{q} refers to the quiescent current of the booster circuit 1810 which is the current flowing in the internal components of the booster circuit 1810 in the no-load state. The constant η and the quiescent current I_{q} may be determined based on an efficiency curve of the booster circuit 1810 working at the static operating point. The static operating point may ensure that n is essentially unchanged when the output load changes. That is, η may be a constant during the working process.

In some embodiments, the abnormity detection circuit 1840 may include a voltage acquisition subcircuit. An input end of the voltage acquisition subcircuit may be connected with the output end of the filter circuit 1830, and an output end of the voltage acquisition subcircuit may be connected with the processor 1850. In some embodiments, as shown in FIG. 21, the voltage acquisition subcircuit may include a voltage divider resistor R7, a voltage divider resistor R8, and a reverse amplifier. The voltage divider resistor R7 and the voltage divider resistor R8 may be in series. One end of the voltage divider resistor R7 may be connected with the output end of the filter circuit 1830. A connection point of the voltage divider resistor R7 and the voltage divider resistor R8 may be connected with an input end of the reverse amplifier. An output end of the reverse amplifier may be connected with the processor 1850.

In some embodiments, the voltage divider resistor R7 and the voltage divider resistor R8 may divide the voltage output by the filter circuit 1830. When the feedback proportional coefficient is large, a voltage across the voltage divider resistor R8 may be relatively small, which is convenient for the processor 1850 to analyze and process the voltage output by the filter circuit 1830 based on the voltage across the voltage divider resistor R8. Specific implementation of the voltage divider resistor R7 and the voltage divider resistor R8 may refer to the descriptions of the voltage divider resistor R5 and the voltage divider resistor R6, which is not be repeated herein.

The reverse amplifier may be an electronic device that changes an input direction and amplifies an input signal. In some embodiments, the reverse amplifier may amplify the voltage across the voltage divider resistor R8, and change a negative voltage across the voltage divider resistor R8 into a positive voltage, so as to facilitate the subsequent processing through the processor 1850.

In some embodiments, the abnormity detection circuit 1840 may include an analog-to-digital converter ADC. The output end of the voltage acquisition subcircuit and the output end of the current acquisition subcircuit may be connected with the input end of the processor 1850 through the analog-to-digital converter ADC. The analog-to-digital converter ADC may be an electronic device that converts an input analog signal into a digital signal. In some embodiments, the analog-to-digital converter ADC may convert the output of the voltage acquisition subcircuit into a voltage value, and convert the output electrical energy of the current acquisition subcircuit into a current value, so as to facilitate the subsequent processing through the processor 1850.

The processor 1850 may be an electronic device that is configured to process data. In some embodiments, the processor 1850 may determine whether the load 200 is working in an abnormal state based on the parameters (e.g., the current of the input power supply, the voltage output by the filter circuit 1830, etc.) of the input electrical energy. In some embodiments, the processor 1850 may determine whether the load 200 is working in an abnormal state based on an abnormal state (e.g., the voltage output by the booster circuit 1810 is too large, the current output by the booster circuit 1810 is too large, etc.) of the booster circuit 1810. For example, if the processor 1850 determines that the booster circuit 1810 is abnormal, the output end of the processor 1850 may output a high electrical level to the enable end EN of the controller U1, so that the controller U1 may stop working to avoid damage to the load 200 of the high-voltage power supply due to the abnormal operation of the booster circuit 1810.

The possible beneficial effects of the embodiments of the present disclosure may include but may not be limited to: (1) a first power supply may be converted into a forward power supply and a negative power supply through the processing unit, so that the forward power supply and the negative power supply may be provided at the same time; (2) a signal processed by a power supply apparatus and an output electrical power may avoid to affect a normal working state of a load in an effective frequency range by setting a resonant frequency of a resonant circuit in the processing unit outside the effective frequency range of the load, thereby reducing the EMI of the power supply apparatus to the load; (3) the current of the forward power supply and the current of the negative power supply may be stable by converting the electrical energy based on the resonant circuit; (4) a same rectification and filtering manner may be used to separately process the electric energy output by the resonant circuit based on a symmetrical structure of a rectifier and filter circuit, thereby improving the symmetry of the forward power supply and the negative power supply, and avoiding the generation of second harmonics that cause EMI to the load; (5) the stability of the load power supply may be effectively improved through a multi-feedback control manner of a first feedback circuit and a second feedback circuit; (6) the power drop of a booster circuit may be avoided and the stability of the output electrical energy of the high-voltage power supply may be improved by compensating the booster circuit based on the input of the booster circuit through a compensation circuit, so that the load may be in a normal working state; (7) the quality of the output of the high-voltage power supply may be improved, the noise floor of the imaging of the load may be reduced, and the quality of the imaging of the load may be improved by filtering the ripples in the output electrical energy of the booster circuit through a filter circuit.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by +20%. Correspondingly, in some embodiments, the numerical parameters used in the description and claims are approximate values, and the approximate values may be changed according to the required characteristics of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

1. An apparatus for supplying power, comprising a power supply circuit and a processing unit, wherein
the power supply circuit is connected with an electric energy input end of the processing unit, and the power supply circuit provides a first power supply for the processing unit; and
a forward voltage output end of the processing unit provides a forward power supply, and a backward voltage output end of the processing end provides a negative power supply,
wherein the processing unit includes a resonant circuit, a first switching frequency of the processing unit includes a resonant frequency of the resonant circuit, and the resonant frequency is outside an effective frequency range of a load of the apparatus for supplying power.

2. The apparatus of claim 1, wherein the resonant frequency being outside the effective frequency range of the load includes:
the resonant frequency being outside the effective frequency range of the load and an nth harmonic frequency range of the effective frequency range of the load; and/or
an nth harmonic of the resonant frequency being outside the effective frequency range of the load and the nth harmonic frequency range of the effective frequency range of the load.

3. The apparatus of claim 2, wherein the resonant circuit includes a resonant capacitor bank and an inductor, the resonant capacitor bank includes one or more resonant capacitors, and the resonant capacitor bank is connected in series with the inductor.

4. The apparatus of claim 3, wherein the resonant capacitor bank includes a first resonant capacitor subset and a second resonant capacitor subset, one end of the first resonant capacitor subset is connected with one end of the second resonant capacitor subset, another end of the first resonant capacitor subset is configured to receive a biasing voltage, and another end of the second resonant capacitor subset is grounded.

5. The apparatus of claim 4, wherein the first resonant capacitor subset and the second resonant capacitor subset are symmetrically arranged with a connection end of the first resonant capacitor subset and the second resonant capacitor subset as a center.

6. The apparatus of claim 5, wherein a difference in capacitance between the first resonant capacitor subset and the second resonant capacitor subset is less than or equal to 5% of either capacitance of the first resonant capacitor subset or the second resonant capacitor subset.

7. The apparatus of claim 3, wherein the processing unit further includes a rectifier and filter circuit, an input end of the rectifier and filter circuit is connected with an output end of the resonant circuit, the rectifier and filter circuit is symmetrically arranged with the resonance circuit as a center, a first output end of the rectifier and filter circuit provides the forward power supply, and a second output end of the rectifier and filter circuit provides the negative power supply.

8. The apparatus of claim 3, wherein the resonant circuit further includes a switch and a driver (U1), an electric energy input end of the driver (U1) is connected with the power supply circuit, the driver (U1) is configured to receive a drive signal and control the switch, the switch is connected with the power supply circuit, and the switch is configured to receive the first power supply; and
a difference between a frequency of the drive signal and the resonant frequency is within a preset range.

9. The apparatus of claim 1, the apparatus further includes a first feedback circuit, wherein
the first feedback circuit is configured to receive and output the forward power supply, the negative power supply, and a reference voltage to a processor (MCU), so that the processor (MCU) transmits a first enable signal to the processing unit (120).

10. The apparatus of claim 9, the apparatus further includes a second feedback circuit, the second feedback circuit includes a first comparator (U5) and a second comparator (U6), wherein
the first comparator (U5) outputs a first comparison result according to the forward power supply and the reference voltage, the second comparator (U6) outputs a second comparison result according to the negative power supply and the reference voltage; and
the second feedback circuit transmits a second enable signal and a third enable signal to the processing unit according to the first comparison result and the second comparison result.

11. The apparatus of claim 10, wherein the processing unit-controls a working state of the resonant circuit according to the first enable signal, the second enable signal, and the third enable signal.

12. The apparatus of claim 10, wherein the apparatus further includes a hysteresis resistor (R10), the hysteresis resistor (R10) is arranged between a forward input end of the second comparator (U6) and an output end of the second comparator (U6).

13. A medical device, comprising: a detector and an apparatus for supplying power, wherein the apparatus comprises a power supply circuit and a processing unit
the power supply circuit is connected with an electric energy input end of the processing unit, and the power supply circuit provides a first power supply for the processing unit; and
a forward voltage output end of the processing unit provides a forward power supply, and a backward voltage output end of the processing end provides a negative power supply,
wherein the processing unit includes a resonant circuit, a first switching frequency of the processing unit includes a resonant frequency of the resonant circuit, and the resonant frequency is outside an effective frequency range of a load of the apparatus for supplying power.

14. A high-voltage power supply, comprising: a booster circuit and a compensation circuit, wherein
an input end of the booster circuit is connected with an input power supply, and an output end of the booster circuit is configured to provide output electrical energy;
an input end of the compensation circuit is at least connected with the input end of the booster circuit;
an output end of the compensation circuit is at least connected with a reference end of the booster circuit, and the compensation circuit is configured to adjust the output electrical power of the booster circuit according to a voltage of the input power supply.

15. The high-voltage power supply of claim 14, wherein the output electrical energy of the booster circuit is controlled by input electrical energy of the reference end of the booster circuit.

16. The high-voltage power supply of claim 15, wherein the reference end of the booster circuit includes a reference voltage, an equivalent resistance to ground of the reference voltage corresponds to a voltage output by the booster circuit.

17. The high-voltage power supply of claim 16, wherein the compensation circuit includes a current acquisition subcircuit and a compensation resistor, an input end of the current acquisition subcircuit is at least connected with the input end of the booster circuit, an output end of the current acquisition subcircuit is at least connected with one end of the compensation circuit, and another end of the compensation circuit is at least connected with the reference end of the booster circuit.

18. The high-voltage power supply of claim 17, the compensation circuit includes a first voltage divider resistor and a second voltage divider resistor in series, wherein
an input end of the first voltage divider resistor is at least connected with the output end of the booster circuit, and a connection point of the first voltage divider resistor and the second voltage divider resistor is at least connected with the reference end of the booster circuit.

19. The high-voltage power supply of claim 18, wherein a working state of the booster circuit is controlled by the first voltage divider resistor and the second voltage divider resistor, when the booster circuit is in a normal working state, a sum of a current output by the booster circuit and a current when the booster circuit is in a no-load wording state corresponds to a current of the input power supply.

20. The high-voltage power supply of claim 17, the current acquisition subcircuit includes an operational amplifier and an acquisition resistor, the acquisition resistor is arranged with the input end of the booster circuit, a first input end and a second input end of the operational amplifier is connected with both ends of the acquisition resistor respectively, and an output end of the operational amplifier is at least connected with the compensation resistor.

21-25. (canceled)
